(19) United States
(12) Patent Application Publication
(10) Pub. No.: US 2025/0381274 A1
(43) Pub. Date: Dec. 18, 2025

(54) METHODS FOR CROSSLINKING OF COLLAGENOUS TISSUE

(71) Applicant: The Trustees of Columbia University in the City of New York, New York, NY (US)

(72) Inventors: Sinisa Vukelic, New York, NY (US); Jiashuai Fan, New York, NY (US)

(21) Appl. No.: 19/171,796

(22) Filed: Apr. 7, 2025

Related U.S. Application Data
(63) Continuation of application No. PCT/US2023/076460, filed on Oct. 10, 2023.
(60) Provisional application No. 63/378,725, filed on Oct. 7, 2022.
(60) Provisional application No. 63/386,662, filed on Dec. 8, 2022.

(51) Int. Cl. A61K 41/00; A61F 9/00; A61F 9/008; A61N 5/06; A61N 5/067; A61P 27/02

(52) U.S. Cl. CPC A61K 41/0057; A61F 9/0008; A61N 5/062; A61N 5/067; A61P 27/02; A61F 2009/00872; A61F 2009/00893; A61N 2005/0661

(57) ABSTRACT

Methods of crosslinking collagenous tissue include contacting the tissue with a sugar; and illuminating the tissue with any one or more of UV-A light or a femtosecond laser, the illuminating being performed under conditions sufficient to give rise to crosslinking within the tissue is a method of treating a tissue of a cornea. Method of treating a tissue of a cornea include restricting oxygen replenishment of the tissue; contacting the tissue with a sugar; and illuminating the tissue with at least one of UV-A light or a femtosecond laser, the illuminating being performed under conditions sufficient to give rise to crosslinking within the tissue. Methods of treating a collagenous tissue include contacting the tissue with a sugar; and illuminating the tissue with any one or more of UV-A light or a femtosecond laser, the illuminating being performed under conditions sufficient to give rise to crosslinking within the tissue.

### CROSS REFERENCE TO RELATED APPLICATIONS

The present application is a Bypass Continuation Application of PCT Patent Application No. PCT/US2023/076460 (filed Oct. 10, 2023); which claims priority to and the benefit of U.S. patent application No. 63/378,725 (filed Oct. 7, 2022) and U.S. patent application No. 63/386,662 (filed Dec. 8, 2022). All foregoing applications are incorporated herein by reference in their entireties for any and all purposes.

### TECHNICAL FIELD

The present disclosure relates to the field of methods of treating the cornea.

### BACKGROUND

Corneal crosslinking (CxL) is a treatment for treating patients with keratoconus (KCN), a noninflammatory condition in which the cornea progressively thins, weakens, and results in irregular astigmatism, myopia, and protrusion. To CxL corneal stroma, the Dresden protocol is performed: de-epithelized eyes are soaked with riboflavin-5-phosphate (R5P) and then exposed to long wavelength ultraviolet (UVA) light, usually centered at 365 nm. Riboflavin-mediated CxL halts the progression of keratoconus by introducing intra and intermolecular chemical bond formation within the collagen fibril-comprised stromal extracellular matrix (ECM).

Upon activation of ultraviolet photons, the riboflavin molecule generates reactive oxygen species (ROS) through both Type I and Type II mechanisms depending on oxygen availability. Drawbacks of riboflavin-mediated CxL include UV-light's cytotoxic effects on stromal keratocytes and endothelium cells for corneas that are not sufficiently thick and the typical removal of epithelium for easier R5P absorption into the stroma and open access to oxygen for the anterior stroma.

Accordingly, there is a long-felt need in the art for improved methods of cornea treatment, particularly improved methods of effecting CxL.

### SUMMARY

In meeting the described long-felt needs, the present disclosure provides a method of treating a tissue of a cornea, the method comprising: contacting the tissue with a sugar; and illuminating the tissue with any one or more of UV-A light or a femtosecond laser, the illuminating being performed under conditions sufficient to give rise to crosslinking within the tissue.

Also provided is a method of treating a tissue of a cornea, the method comprising: restricting oxygen replenishment of the tissue; contacting the tissue with a sugar; and illuminating the tissue with at least one of UV-A light or a femtosecond laser, the illuminating being performed under conditions sufficient to give rise to crosslinking within the tissue.

Further provided is a method of treating a collagenous tissue, the method comprising: contacting the tissue with a sugar; and illuminating the tissue with any one or more of UV-A light or a femtosecond laser, the illuminating being performed under conditions sufficient to give rise to crosslinking within the tissue.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides exemplary equilibrium modulus and viscoelastic ratio results.

FIG. 2 provides exemplary results related to corneal refractive power change.

FIG. 3 provides exemplary instantaneous modulus, equilibrium modulus, and viscoelastic ratio results.

FIG. 4 provides representative 5 μm indentation stress-relaxation results.

FIG. 5 provides exemplary 860 nm-SHG and 860 nm AF results.

FIG. 6 provides representative images related to the Hypothesis 1A discussion herein.

FIG. 7 provides representative OCT and visual changes related to the Hypothesis 1A discussion herein.

FIG. 8 provides exemplary corneal refractive power change with IR laser and ribose treatment.

FIG. 9 provides representative time history graph for effective refractive power related to the Hypothesis 2A discussion herein.

FIG. 10 provides exemplary Piuma nano-indentation results.

FIG. 11 provides representative 5 μm indentation stress-relaxation results related to the Hypothesis 1B discussion herein.

FIG. 12 provides exemplary confocal microscopy results.

FIG. 13 provides representative images related to the Hypothesis 1B discussion herein.

FIG. 14 provides exemplary visual and OCT changes related to the Hypothesis 2B discussion herein.

FIG. 15 provides exemplary corneal refractive power change with IR laser and ribose treatment.

FIG. 16 provides representative time history graphs for effective refractive power related to the Hypothesis 2B discussion herein.

FIG. 17 provides exemplary results related to the disclosed technology.

FIG. 18 provides exemplary images related to the disclosed technology.

FIG. 19 provides exemplary histology related to the disclosed technology.

FIG. 20 provides exemplary OCT results related to the disclosed technology.

FIG. 21 provides exemplary images related to the disclosed technology.

FIG. 22 provides exemplary results related to the disclosed technology.

FIG. 23 provides exemplary equilibrium modulus data related to the disclosed technology.

FIG. 24 provides exemplary equilibrium modulus related to the disclosed technology.

FIG. 25 provides exemplary equilibrium modulus related to the disclosed technology.

FIG. 26 depicts ROS-Glycation-CxL with ribose and simultaneous mechanical loading. ROS inside the cornea can be generated by, for example, a UV-A light at 365 nm. External loading can be achieved with coverslip application on the anterior cornea.

FIG. 27 depicts (a) an exemplary nano-indentation apparatus, (b) representative load-relaxation response in compression, (c) equilibrium modulus and viscoelastic ratio for ROS-Glycation-CxL (n=10), (d) Dresden CxL (n=9) and corresponding paired controls (**** p<0.0001, *** p<0.001, ns p<0.05).

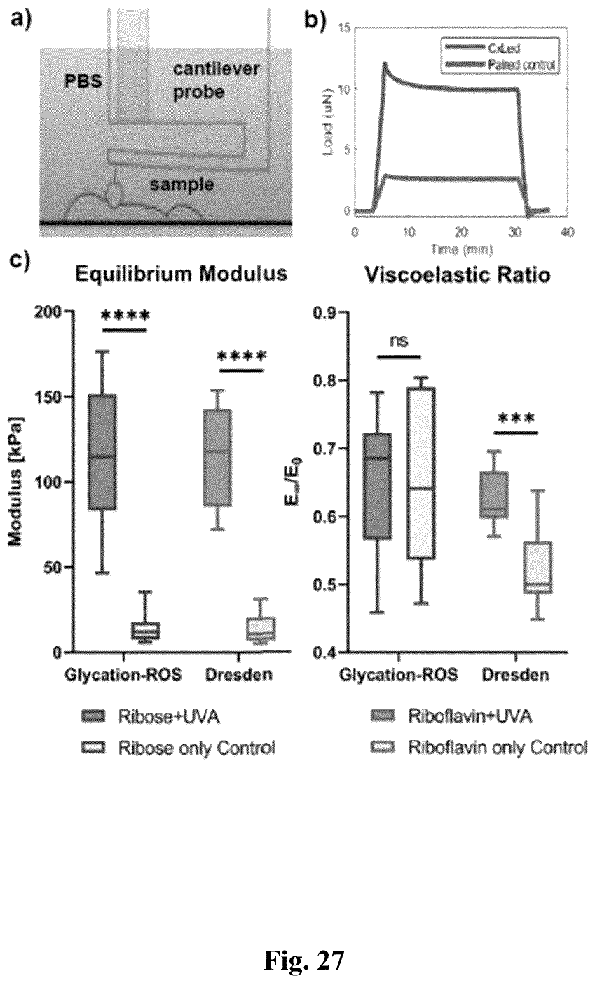

FIG. 28 provides (a) representative OCT images after treatment, and (b) ex vivo rabbit corneal refractive power changes (n=3) from EyeSys topography (* p<0.06, error bars are standard deviations).

FIG. 29 provides (left) representative stress-relaxation curves from crosslinked and control corneal buttons, (middle) statistically significant differences were found between average equilibrium modulus of crosslinked and paired control samples for Group1 A, B, C for ROS-glycation-CxL and D for UVA-R5P-CxL (Dresden protocol) (** p<0.01; *** p<0.001; **** p<0.0001), and (right) viscoelastic ratio calculated from instantaneous and equilibrium modulus. Only the Group1D with UVA-R5-CxL treatment had a significantly different time-dependent elastic response compared to the un crosslinked control (*** p<0.001).

FIG. 30 provides autofluorescence signal intensities from multiphoton imaging for treatment Group 1A, B, C, D. Statistical significance were found for all treated and control pairs. Representative autofluorescence image stacks (612 *612*100 μm) were shown in the XY orientation at α) 1069 nm laser treated corneas, β) UVA lamp treated corneas, y) 400 nm laser treated corneas, and 8) ribose only controls for Group 1A, B C.

FIG. 31 provides second harmonic generation (SHG) signal intensities from multiphoton imaging for treatment Group 1A, B, C, D. Statistical significance were found for all treated and control pairs. Representative SHG image stacks (612 *612*100 μm) were shown in the XY orientation at α) 1069 nm laser treated corneas, β) UVA lamp treated corneas, y) 400 nm laser treated corneas, and 8) ribose only controls for Group 1A, B C.

FIG. 32 provides a time-history of effective refractive power variations over 10 hours for each treatment and control conditions for Group 2A and B. The first point at 0.5 hr represents baseline initial refractive powers before any alterations. The third point represents refractive power after crosslinking and/or loading after 1.5 hr and 3 hr treatments. Shaded areas are standard deviations for time histories from all samples. Also provided is the average change between the initial and third timepoint, and between the initial and final effective refractive power measurements for paired corneas in each group. Significant immediate and sustained refractive power drop differences were found between crosslinked and control pairs (** p<0.01; **** p<0.0001). Error bars are standard deviations.

FIG. 33 provides (left) representative optical coherence tomography (OCT) at the apical cornea for each treatment and control conditions. The time point at 0.5 hr represents the baseline cornea shape before any alterations. The time point at 1.5 hrs or 3 hrs represents the cornea shape after crosslinking and/or loading. The time point at 10 hrs represents the final cornea shape; and (right) the average change between the initial and third timepoint, and between the initial and final apical thickness measurements from OCT the paired corneas in each group. Except for the immediate thickness different for the 3 hr loading in Group 2B, no significant immediate and sustained corneal apical thickness differences were found between crosslinked and control pairs (* p<0.05). Error bars are standard deviations.

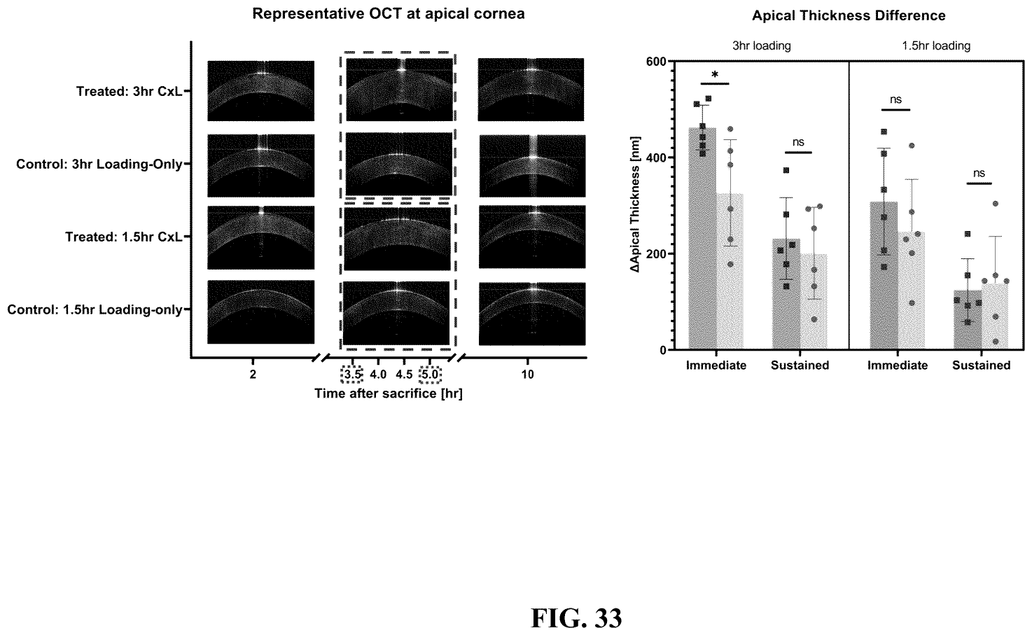

FIG. 34 provides equilibrium modulus and viscoelastic ratio from nano-indentation for Group 2C. Between the 1.5 hr and 3 hr treatments, there is no different in equilibrium modulus; however, there is a significant difference between the time-dependent viscoelastic ratio.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

### Disclosure-I

It was hypothesized that advanced glycation end products-mediated CxL

(Glycation-CxL), a non-enzymatic cross-linking mechanism via the Maillard reaction, can be a viable alternative to existing corneal treatments if the process is accelerated by reactive oxygen species (ROS). Under normal physiological metabolic rates, the AGE-CxL and the stiffening of tissue could take weeks or months, but the process is accelerated and potentiated by reactive oxygen species (ROS) introduced by oxidative stress conditions or by photochemical effects. The free radicals can aid the molecular rearrangement of the sugars to enable reactions between the carbohydrate functional groups and the amino acids in collagen fibrils.

As illustrated here, one proposed ROS-Glycation-CxL employs ribose to initiate protein glycation and does not require oxygen presence. Being a small molecule (150.13 Da), ribose can penetrate the epithelium, which eliminates the need for painful epithelium removal. Further, oxygen independence allows for combining CxL with external mechanical loading, which has utility in reshaping of the corneal curvature.

It has been shown on ex vivo rabbit eyes that a localized, narrow, apical mechanical loading and simultaneous riboflavin mediated-CxL can be utilized for corneal steepening as a treatment for hyperopia. However, if corneal flattening is desired for treating myopia, the apical cornea must be pressed against a wider surface, such as a coverslip; this loading profile blocks the oxygen supply, rendering the Dresden protocol ineffective. Unlike the riboflavin-mediated CxL, glycation and cross-linking of proteins by pentoses can proceed efficiently without oxygen. Finally, ribose (150.13 Da) might penetrate corneal epithelium more easily than riboflavin (376.36 Da) and riboflavin-5-phosphate (456.3 Da) because smaller molecules have higher permeability through corneal epithelium. With its oxygen-independence mechanism, ribose-mediated CxL has applicability to trans-epithelial CxL modalities.

Here, we hypothesize that ROS-accelerated, glycation-mediated CxL can achieve oxygen-independent corneal stiffening for keratoconus treatment and other applications. In addition, we examine the simultaneous application of ROS glycation-CxL and mechanical loading for corneal flattening toward non-invasive vision correction.

### Glycation/Millard Reaction Background

Through glycosylation and glycation, reducing sugars and collagen can react and form collagen crosslinks through the Millard reaction and advanced glycation product (AGE). Millard reactions under physiological conditions are, however, slow. Crosslinking of collagens using reducing sugars such as glucose usually takes days in the incubation chamber. The Millard reaction depends on the temperature and hydration level of the tissue. As explained here, a pentose (e.g. ribose)-initiated reaction (in contrast to a hexose, e.g. glucose) can proceed efficiently without oxygen.

### Ribose and Sugar-Related Collagen Crosslinking in Cornea

The Dresden protocol, or UVA-Riboflavin CXL, is an FDA approved corneal crosslinking protocol to treat keratoconus. The method requires oxygen for the maximum stiffening effect on the pathetically softened cornea. There are clinical reports showing inconclusive evidence on the possible protective effect of diabetes (excessive sugar in cornea from tear and anterior chamber) against the progression of Keratoconus. It has been shown that AGE plays a role in the Dresden protocol. Additionally, the crosslinking introduced by the Dresden protocol and Millard Reaction in cornea are both considered non-enzymatic crosslinking. Without being bound to any particular theory or embodiment, the reaction process can be accelerated by Reactive Oxygen Species (ROS), which rapidly cleave the sugars for molecular rearrangement in glycation.

### Potential Photochemical Activation of Ribose:

Ultra Violet light is able to generate reactive oxygen species. UV light by itself is also used for crosslinking of collagenous tissues or constructs. It has been shown that there is a synergetic effect between glucose and UV-C light on the crosslinking of collagen gels.

### Vision Correction Potential for CXL Using Ribose

As explained herein, evidence showed that simultaneous mechanical loading during corneal crosslinking can lead to a more pronounced and possibly permanent shape change. To achieve flattening of the cornea as a treatment for myopia, the effective loading profile can, for example, include pressing the apical cornea against a surface (such as a coverslip, or an ortho-K lens). Such a loading, however, blocks the supply of oxygen, rendering the Dresden protocol ineffective in this scenario. For this reason, we explore here the photochemical activation of ribose for collagen crosslinking through glycation is an ideal candidate for flattening, because the approach can achieve stiffening and reshaping of the cornea without oxygen and accelerated CXL due to light activation through ROS generation.

First, we explored the hypothesis (Hypothesis 1A) Femtosecond laser at 1069 nm as the activation light source+Ribose could achieve corneal CXL.

### Experimental Set Up:

3 rabbits, (n=8 pairs of 3 mm biopsy punched corneal buttons)
Laser Treatments:
Fidelity II, 1069 nm, 450 mW
Focusing: Marker/spark around, not touching the tissue
Objective: ZEISS 40X, NA=0.1463
Z: increments 20 μm, 5 layers (several intervals)
1stlayer 15 μm below surface of focus sparking
XY: 10 μm zigzag grid, 3 mm radius circle
With ˜12 gram compression
Motor speed 2.6 mm/s, full circle treatments: ˜25*5 mins
Sample Preparation:
−3 mm biopsy punch (1BT)
not connected to IV system
Prior to LT: 30 mins in 20% w/w Dextran 500 in PBS
30 mins in 20% w/w ribose in DPBS (˜1.7M)
Post LT: Trypan blue is used to mark endothelium side
Imaged on the same day in DPBS
Overnight storage in DPBS in 4C fridge
3 hrs prior to indentation, in room temp PBS
Piuma nano-indentation:
For each cornea button, the average of up to 16 indentations are acquired as

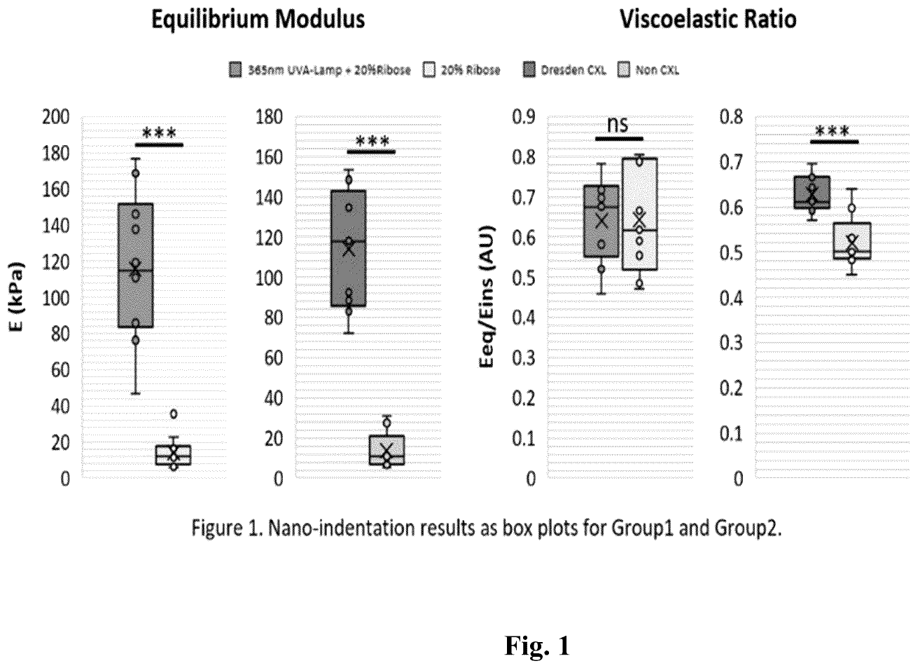

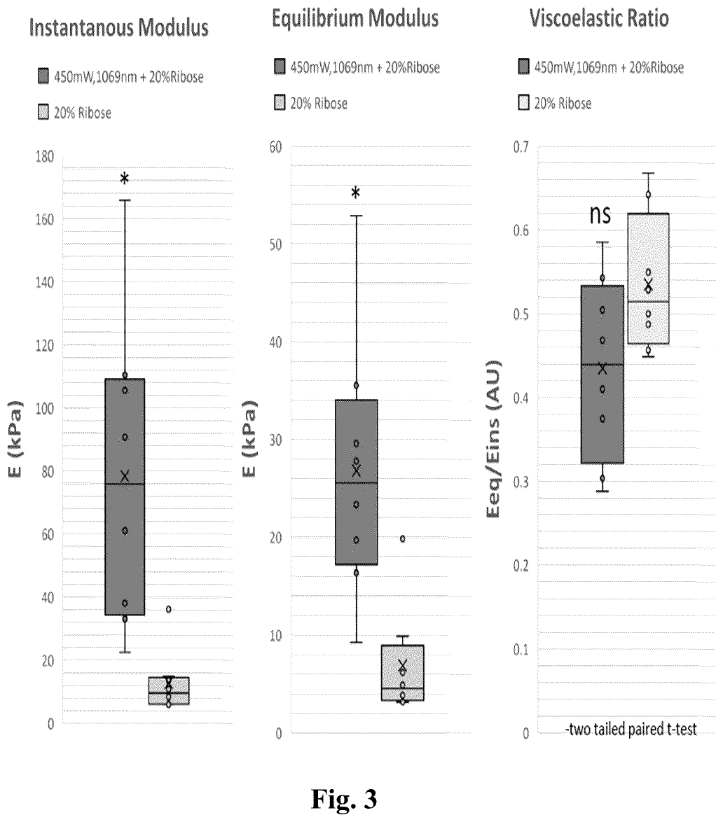

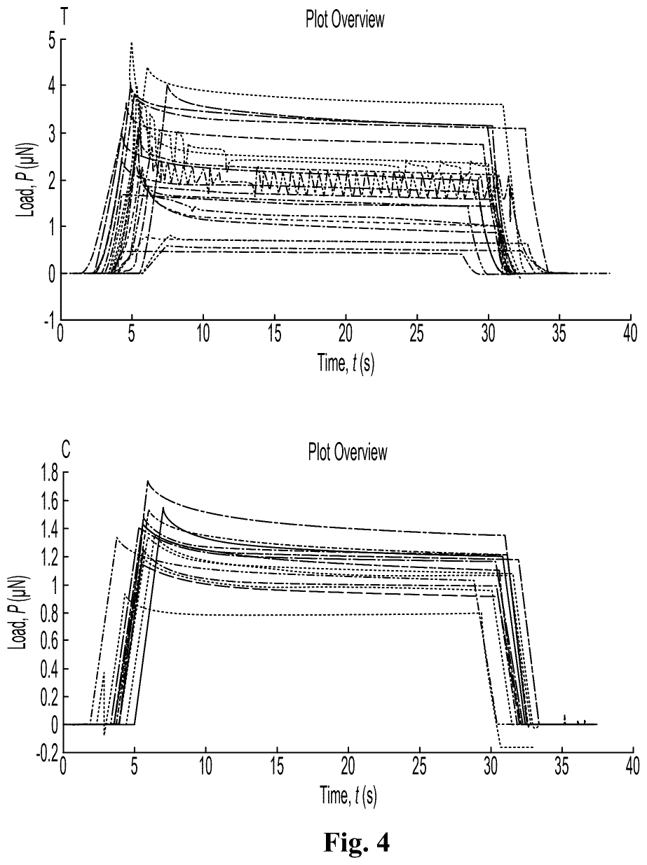

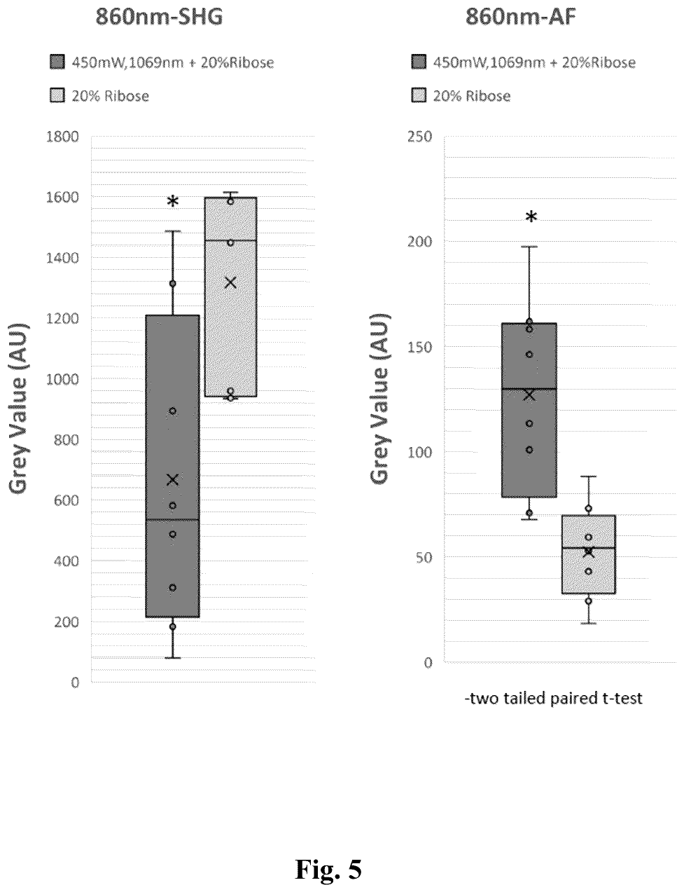

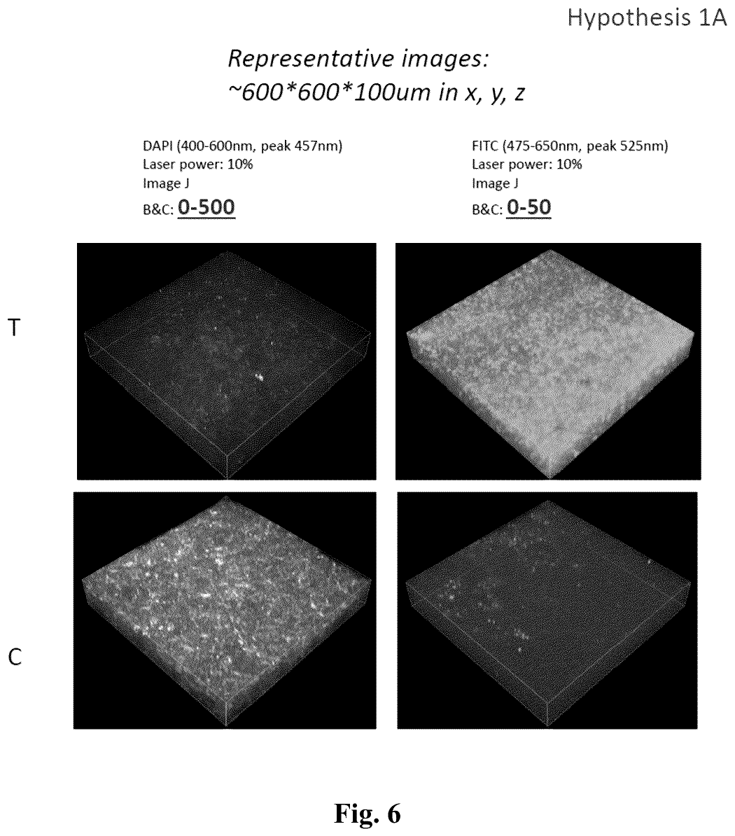

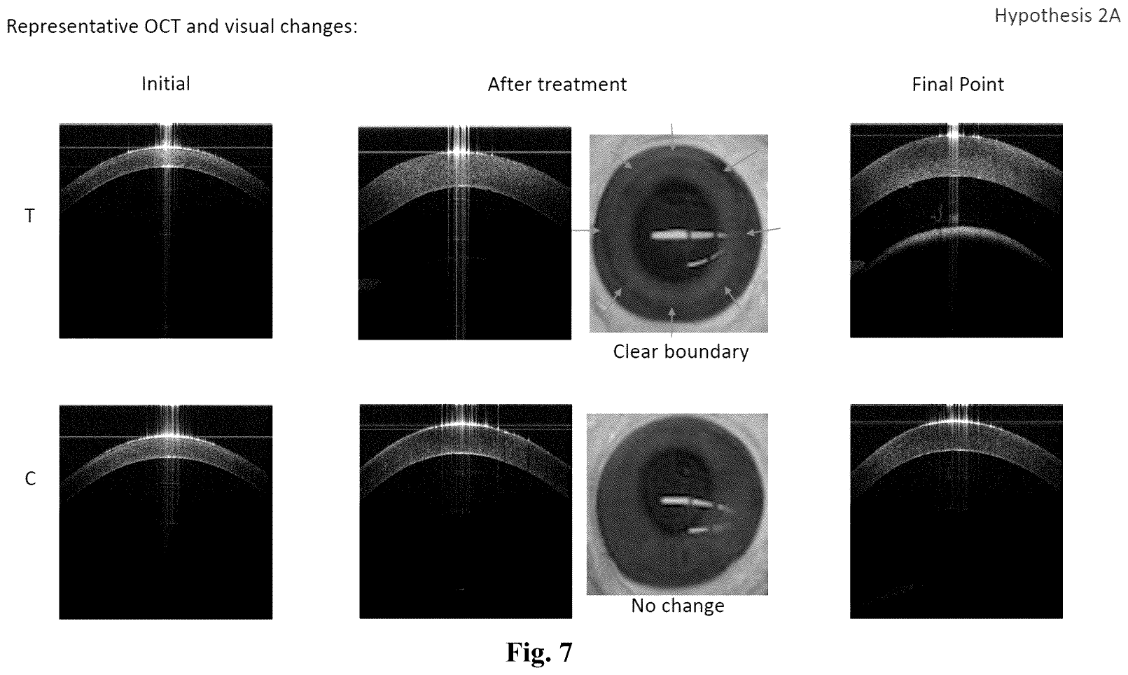

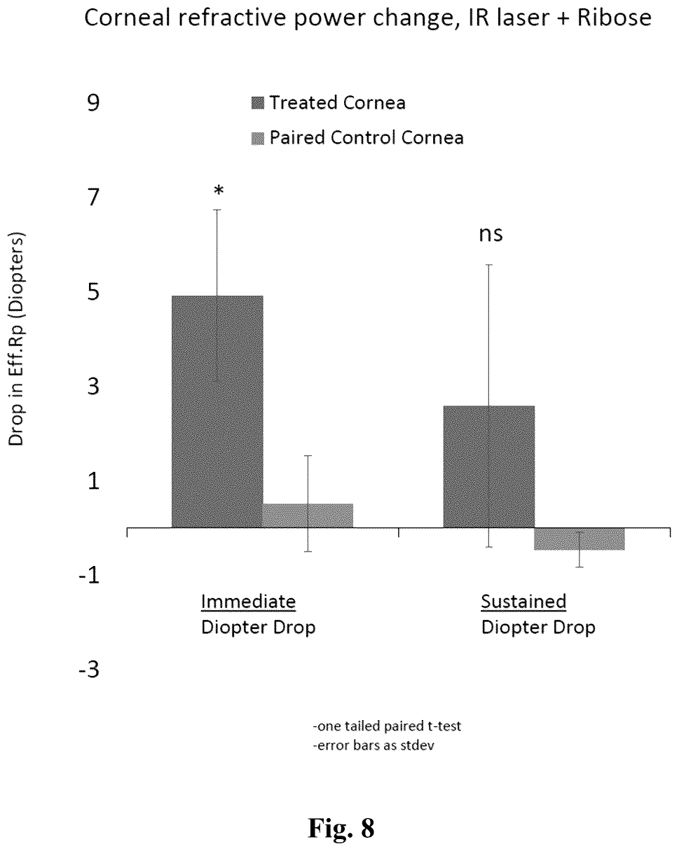

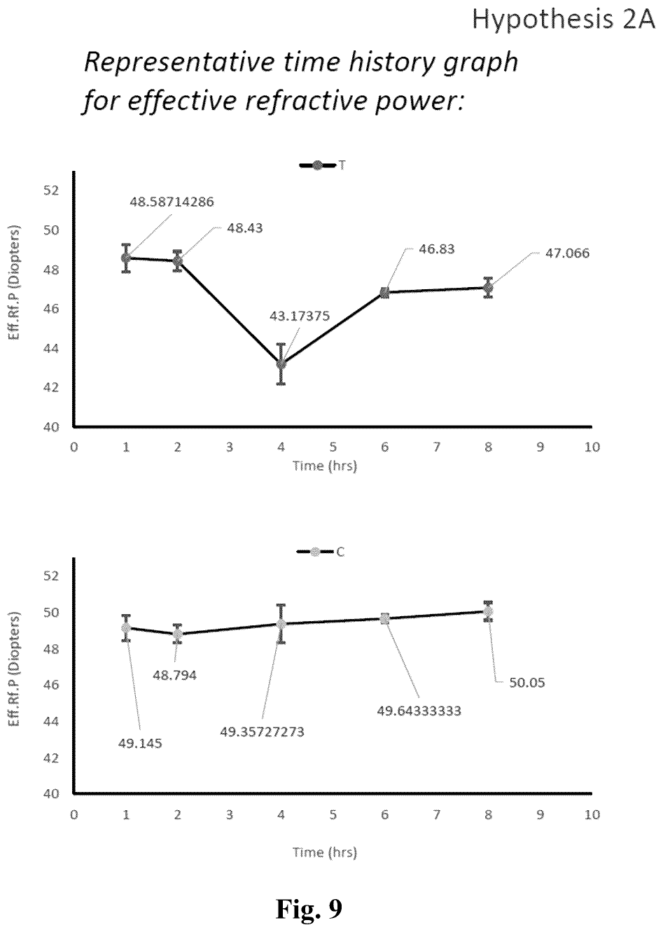

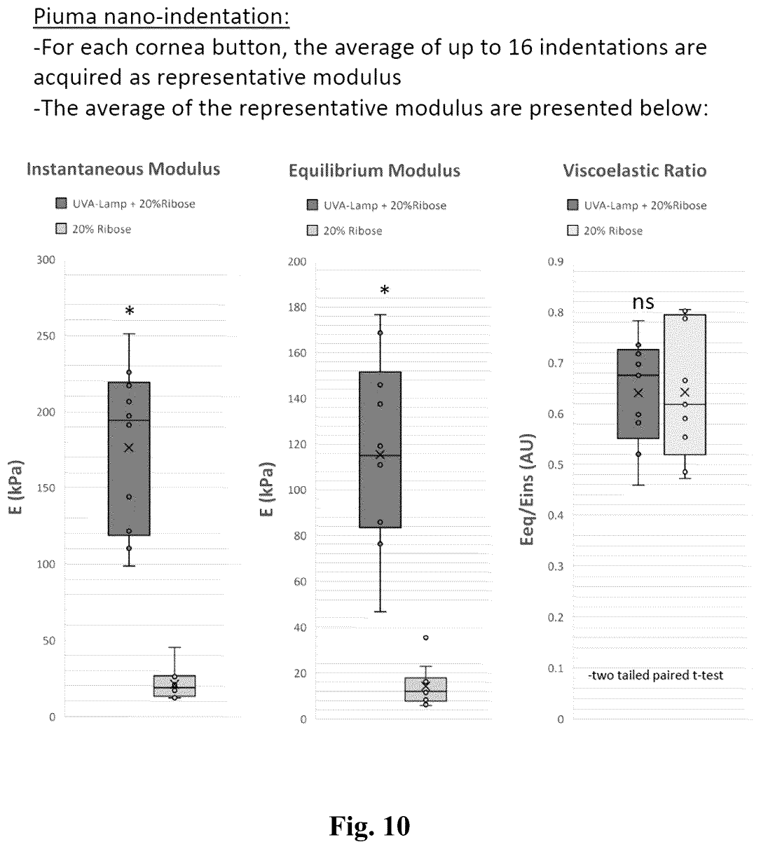

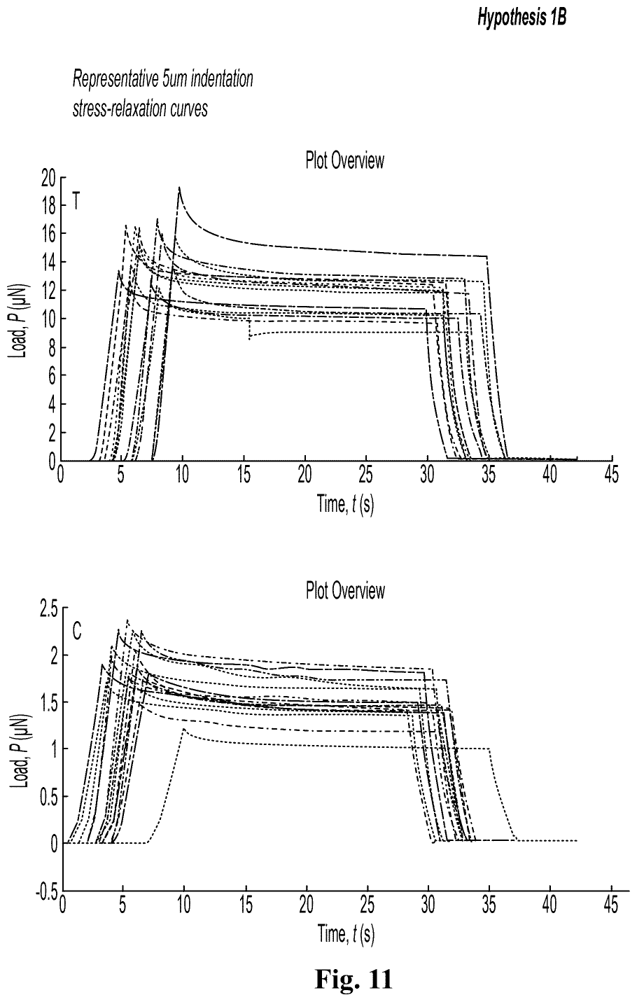

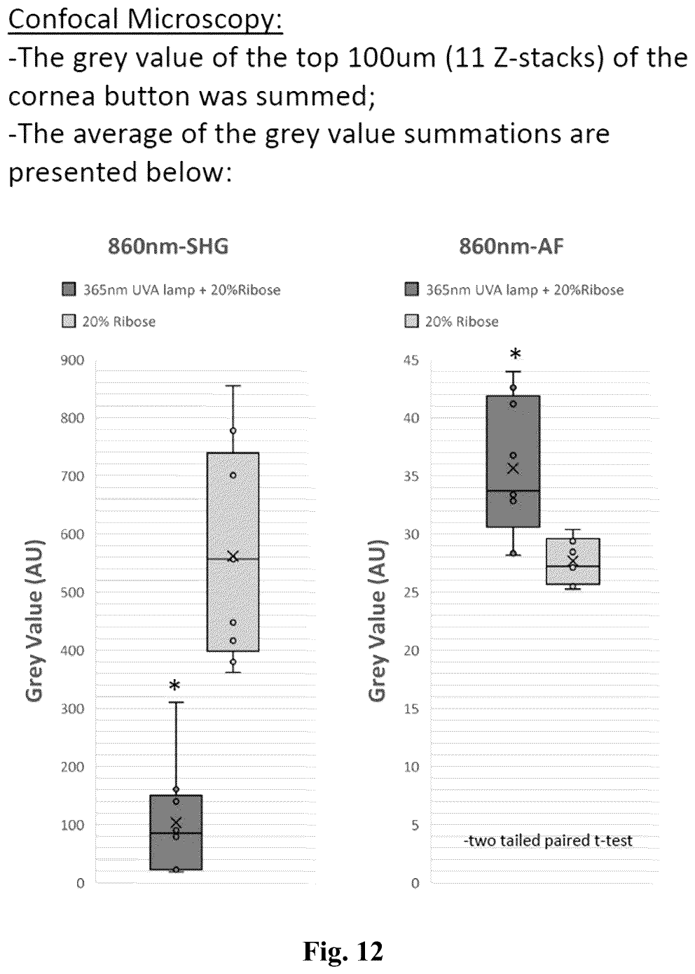

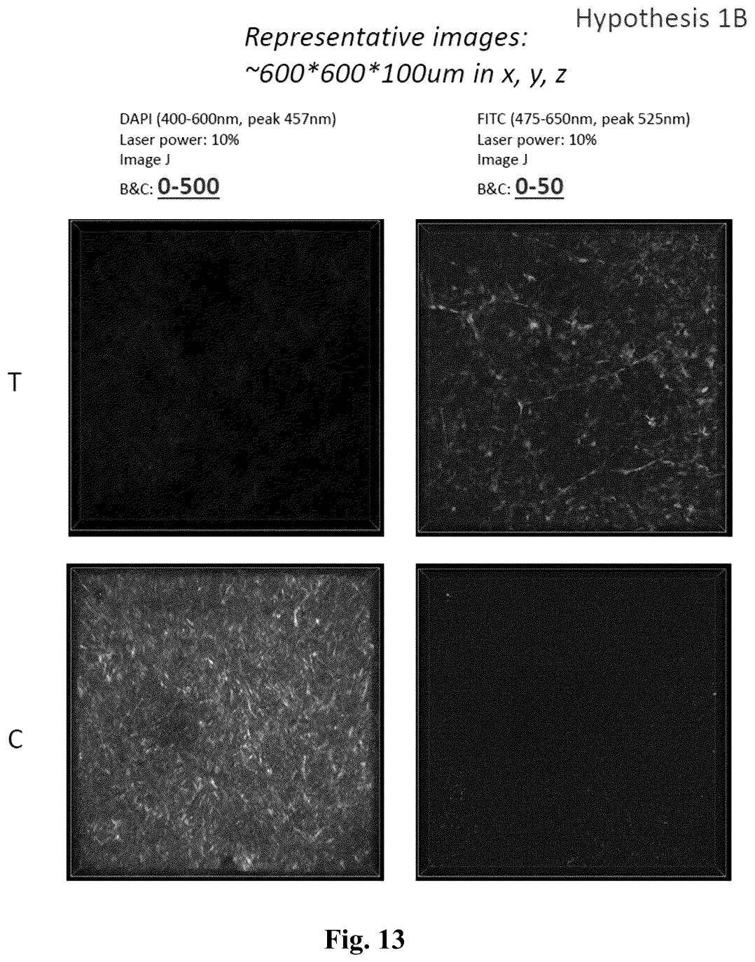

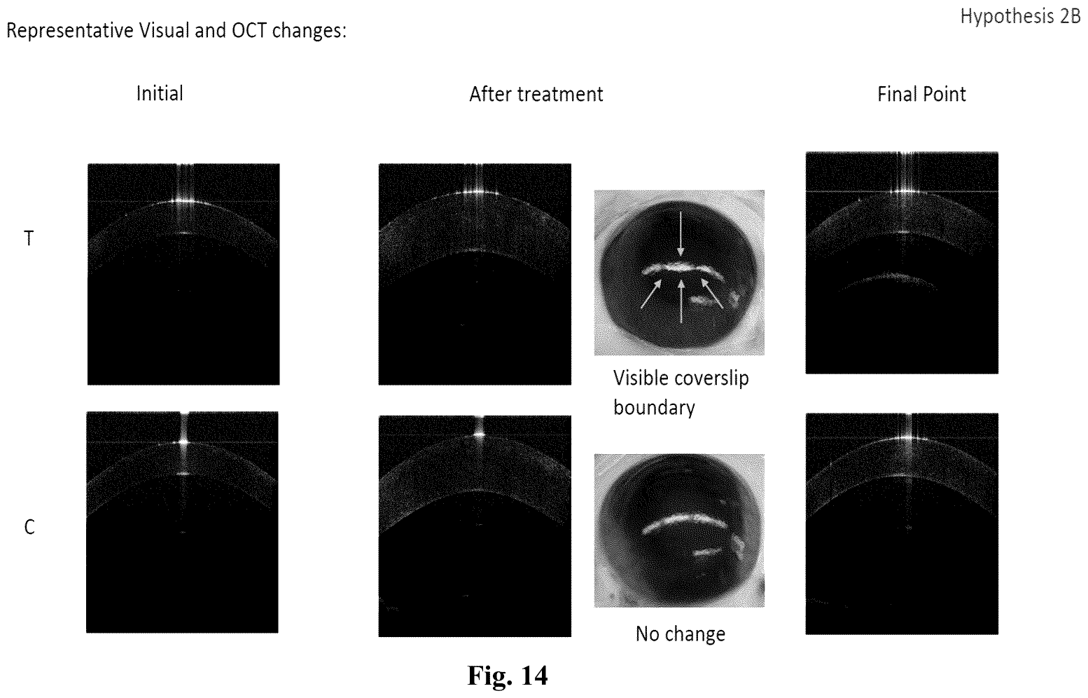

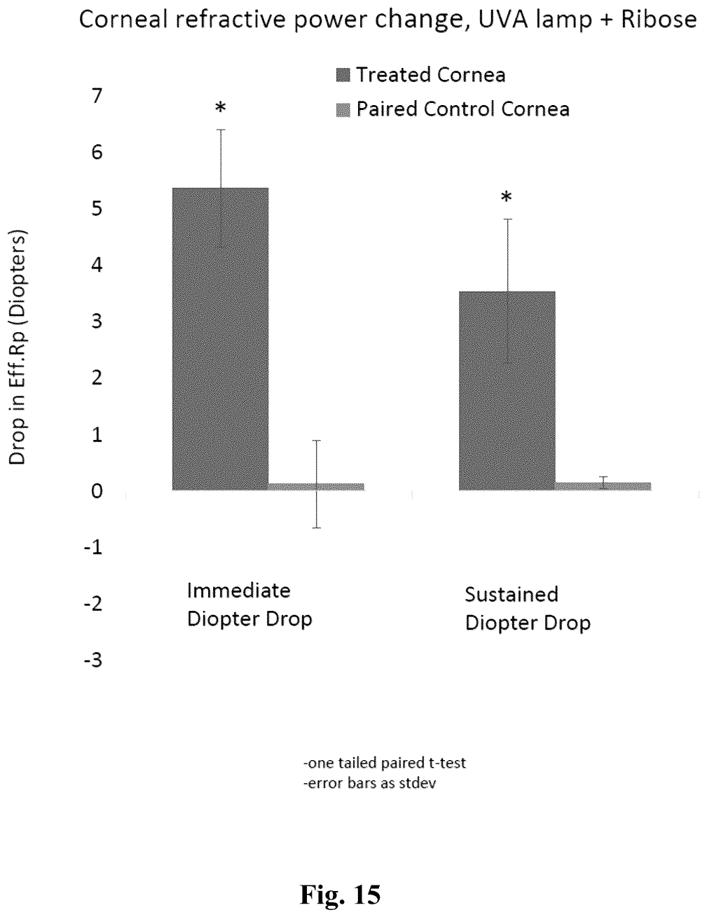

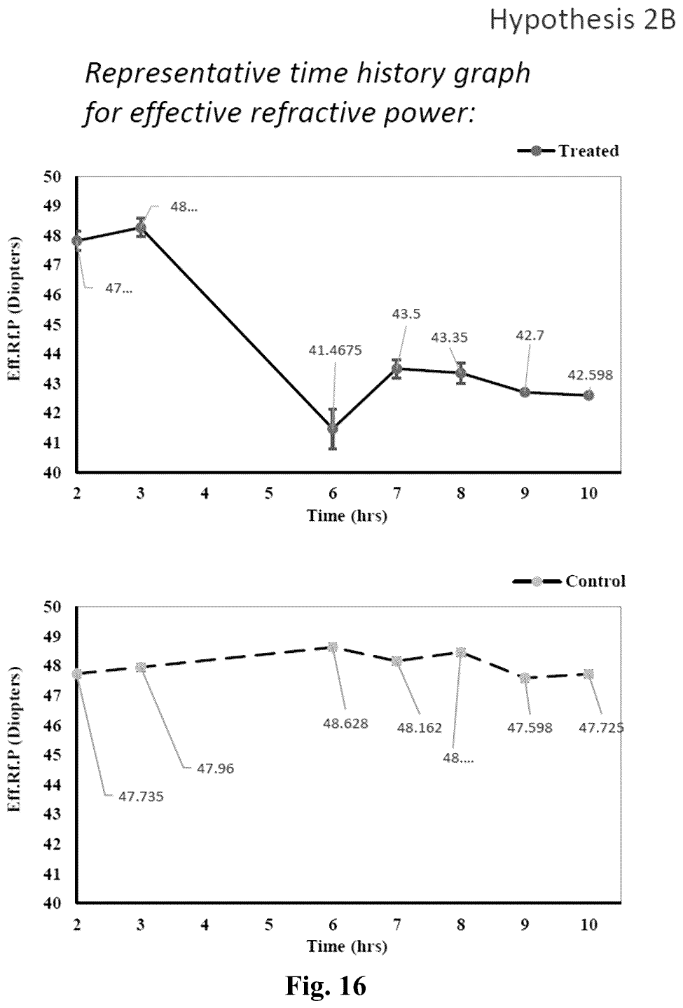

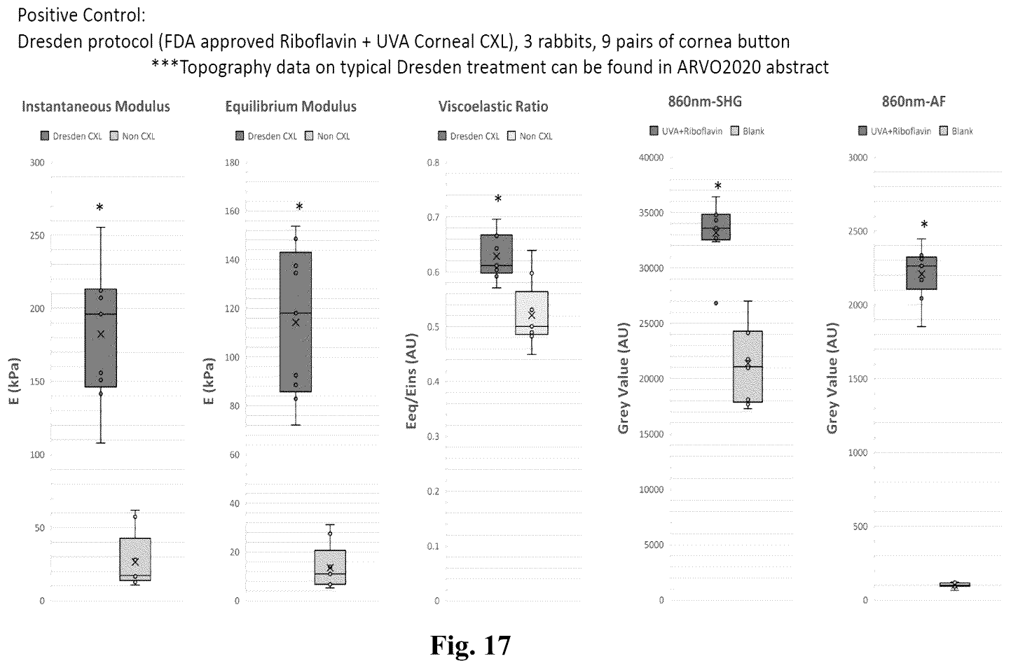

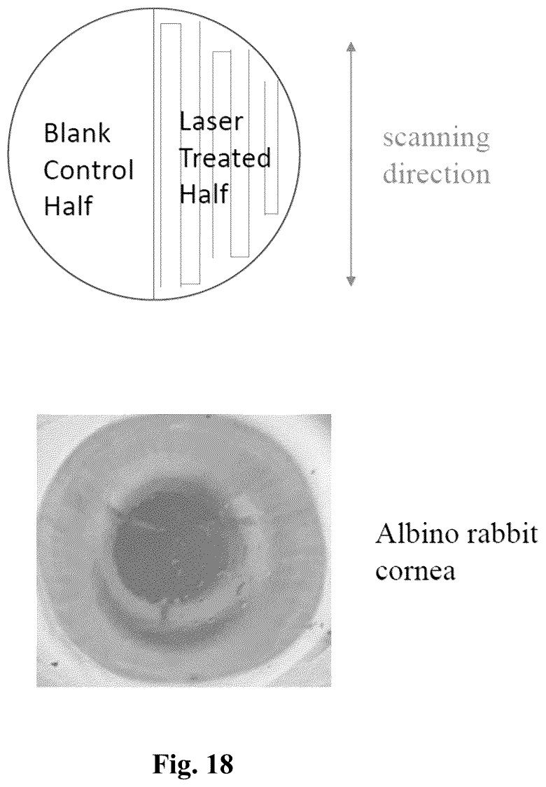

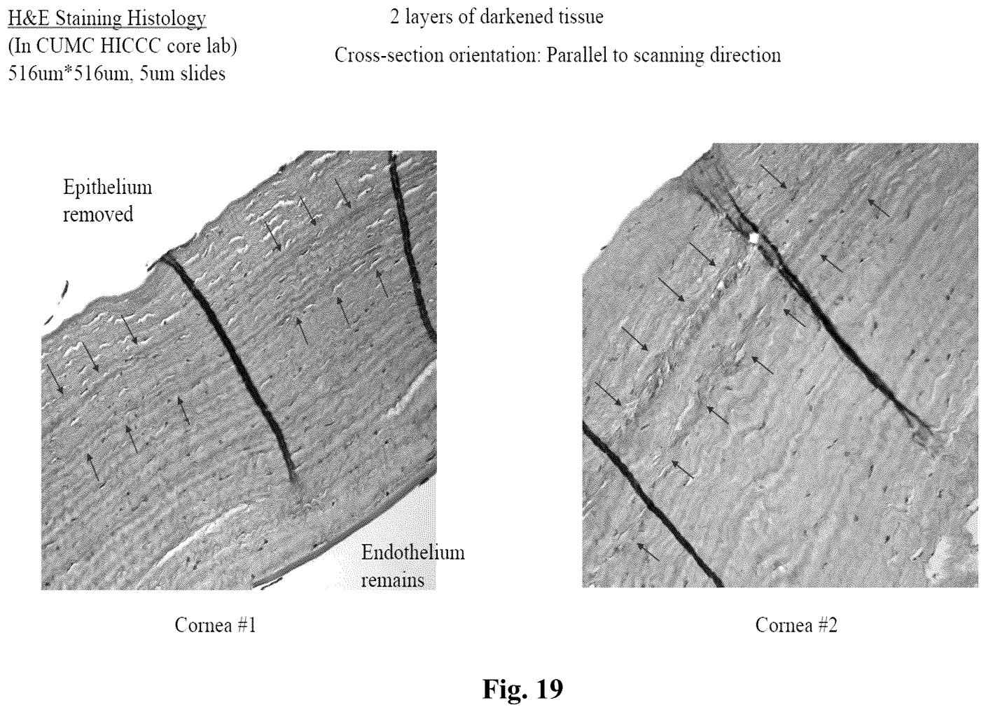

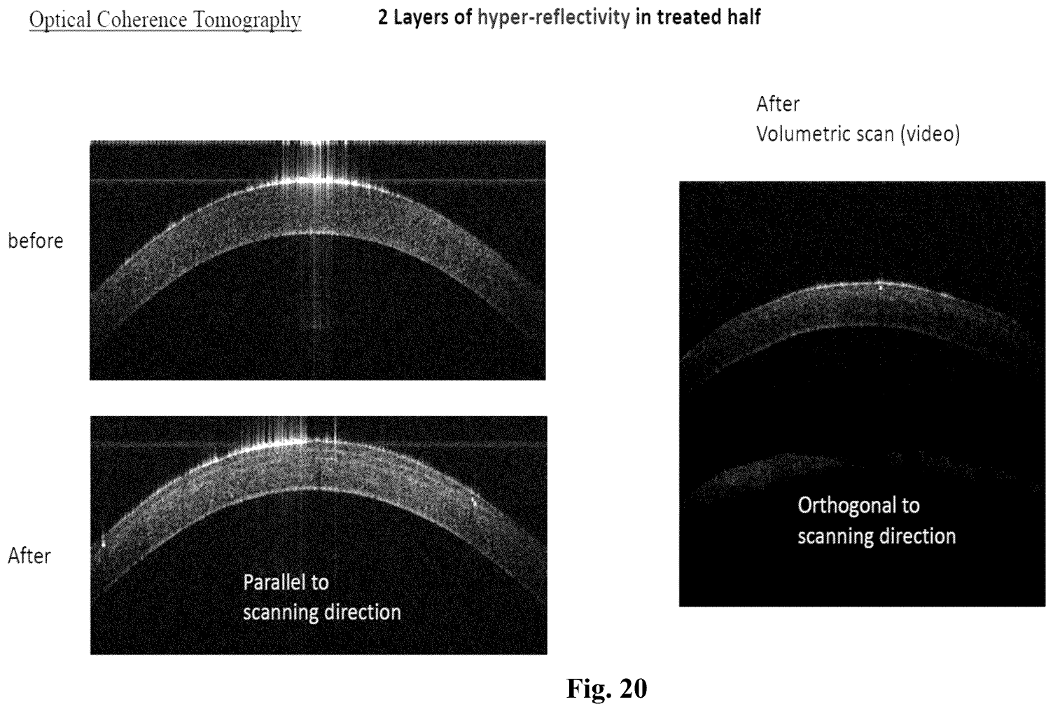

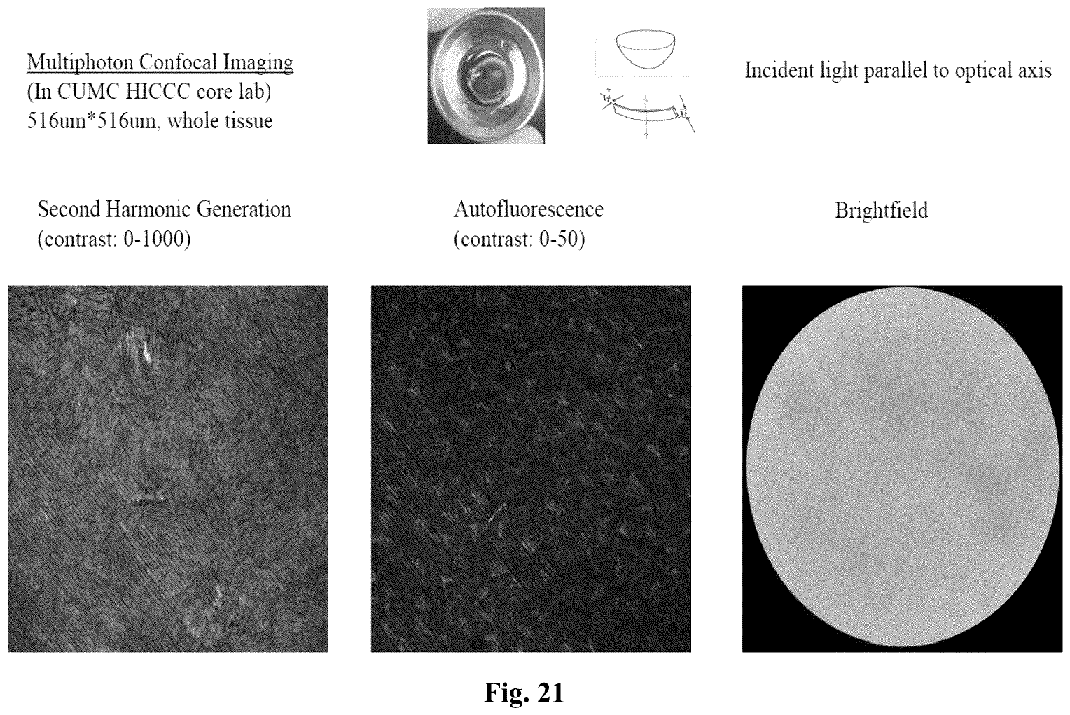

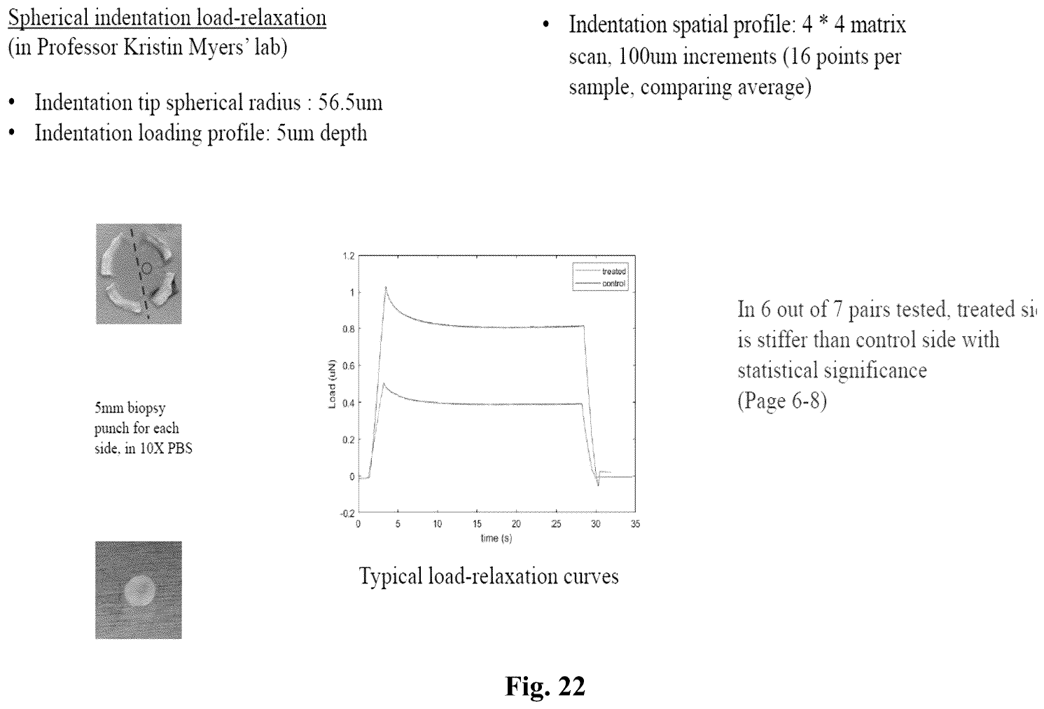

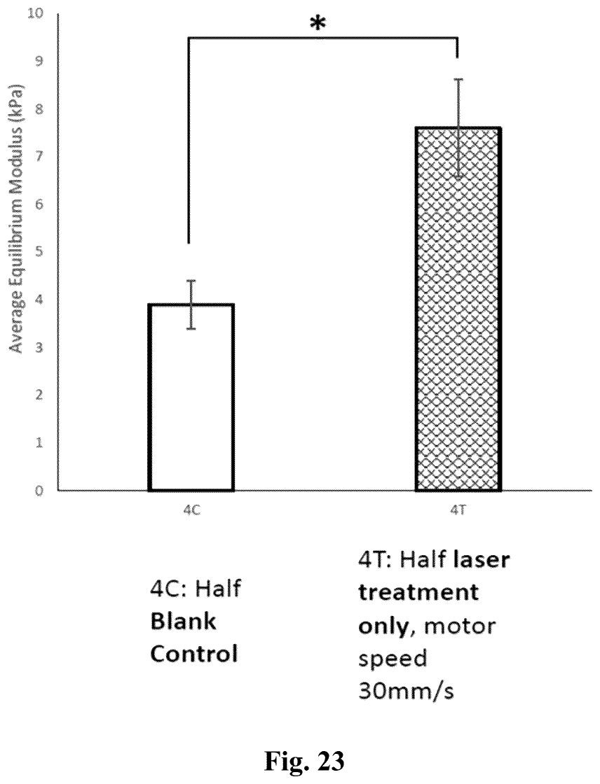

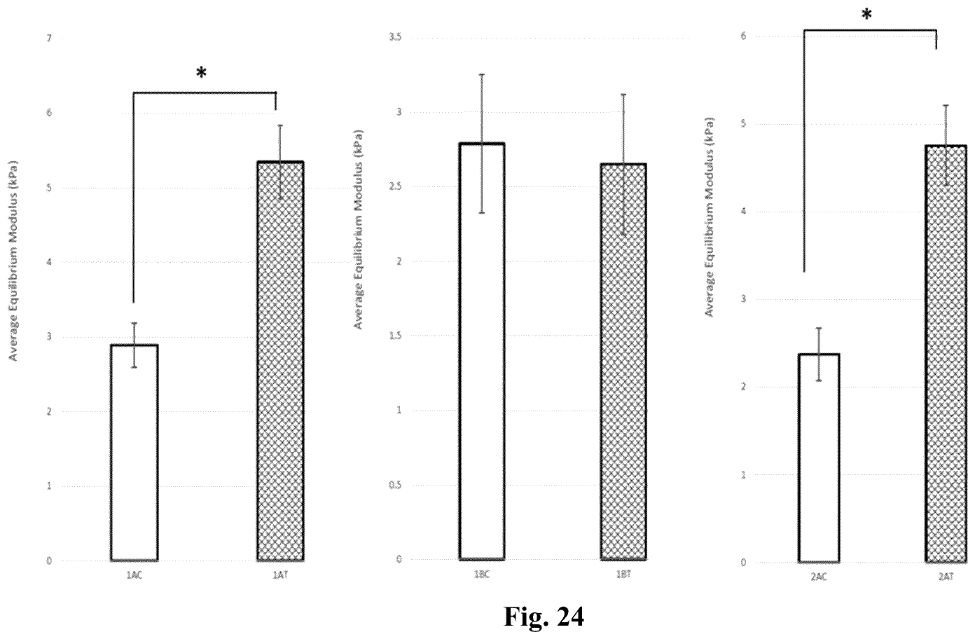

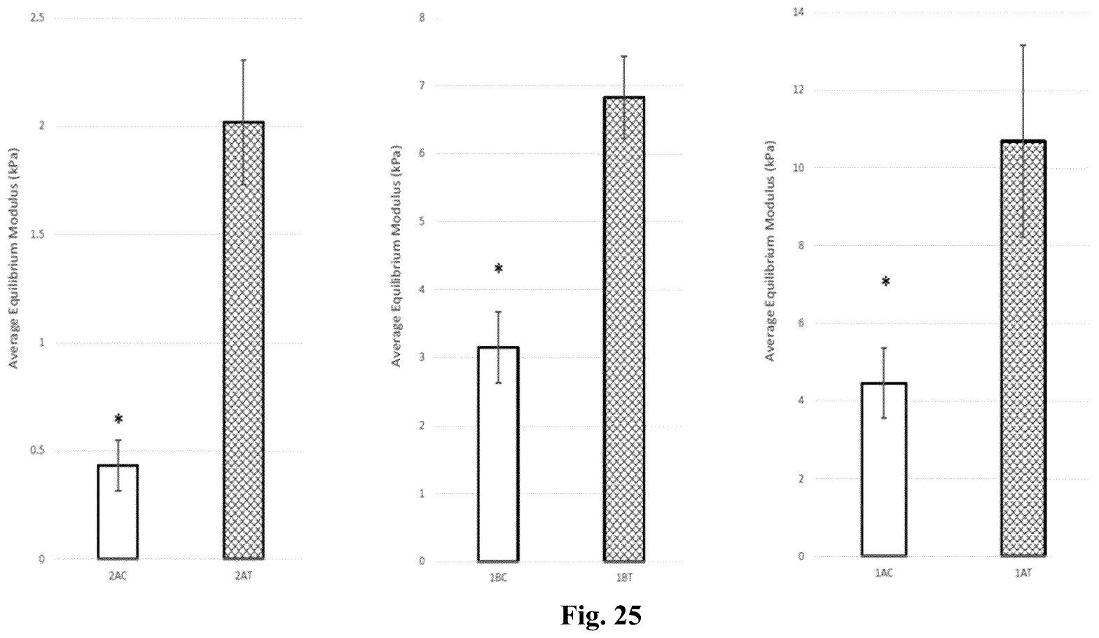

representative modulus
The averages of the representative modulus are presented in appended FIGS. 1-25.

Representative 5 μm indentation stress-relaxation curves are shown.

### Confocal Microscopy:

The grey value of the top 100 μm (11 Z-stacks) of the cornea button was summed;
The average of the grey value summations are presented in appended FIGS. 1-25.

Representative images at ˜600*600*100 μm in x, y, z are shown.

From these experiments, we observed that a femtosecond laser at 1069 nm as the activation light source+Ribose significantly increased both the instantaneous and equilibrium modulus of the cornea. The viscoelastic modulus of the cornea was not significantly changed. The Second harmonic generation signal intensity was decreased, and the autofluorescence signal intensity was increased.

Next, we explored the hypothesis (Hypothesis 2A) that a femtosecond laser at 1069 nm as the activation light source+Ribose could achieve corneal flattening.

### Experimental Set Up:

5 additional rabbits (Paired treated and control for OS/OD)
Sample preparation for ex vivo EyeSys topography:
connected to IV system (˜15 mm H2O)
Epithelium removed
CxL during mechanical deformation introduced by the coverslip
Point1: 1 hr after IV stabilization
Point2: after applying 20% (1.7M) Ribose, then 20% Dextran
Point3: after flattening/treatment (˜2 hrs)
(flattened 8-10 mm)
Point4: ˜1 hr after point3
Point5: last point (might need trypan blue on top)
Laser Treatments:
Fidelity II, 1069 nm, 39fs, 450 mW
Focusing: no (deliberate) spark around touching the tissue
ZEISS 40X, NA=0.1463
Z: increments 20 μm, 5 layers (several intervals)
1 stlayer 15 μm below surface of focus sparking
XY: 10 μm zigzag grid, 3 mm radius circle
Motor speed 2.6 mm/s, full circle treatments: ˜25*5 mins
Representative OCT and visual changes are shown in appended FIGS. 1-25.

From these experiments, we observed a diopter drop immediately after treatment. The change in diopters was not persistent and gradually recovered to the baseline.

Next, we explored the hypothesis (Hypothesis 1B) that using a UVA 365 nm Lamp as the activation light source+Ribose could achieve corneal CXL

### Experimental Set Up:

3 rabbits, (n=10 pairs of 3 mm biopsy punched corneal buttons)
3 mm biopsy punch
not connected to IV system
Treatment Protocol-1 hr submerged in 20% w/v Ribose for all samples
1 hr of UV-A lamp irradiation (control was not under UV)
Post Treatment Trypan blue is used to mark endothelium side
Imaged on the same day in DPBS
Overnight storage in DPBS in 4C fridge
3 hrs prior to indentation, in room temp PBS
Piuma nano-indentation:
For each cornea button, the average of up to 16 indentations are acquired as representative modulus
The average of the representative modulus are presented in appended FIGS. 1-25.

Representative 5 μm indentation stress-relaxation curves are shown

### Confocal Microscopy:

The grey value of the top 100 μm (11 Z-stacks) of the cornea button was summed;
The average of the grey value summations and Representative images at ˜600*600*100 μm in x, y, z are presented in appended FIGS. 1-25.

From these experiments, we observed that 365 nm UVA-Lamp as the activation light source+Ribose significantly increased both the instantaneous and equilibrium modulus of the cornea. The UVA-lamp was more efficient at enhancing mechanical properties compared to the IR laser treatment. The viscoelastic modulus of the cornea was not significantly changed. The Second harmonic generation signal intensity was decreased. This decrease is much more pronounced compared to the IR laser treatment. The autofluorescence signal intensity was increased. The signal increase was smaller in magnitude compared to the IR laser treatment.

Next, we explored the hypothesis (Hypothesis 2B) that using a UVA 365 nm lamp as the activation light source with Ribose could achieve corneal flattening

### Experimental Set Up:

3 additional rabbits (Paired treated and control for OS/OD)
connected to IV system, stabilize for ˜1 hr
Trypan Blue inside the anterior chamber beneath cornea
CxL during mechanical deformation introduced by the coverslip
(˜40%-60% flattened by coverslip from the total diameter)
Treatment Protocol-1 hr submerged in 20% w/v Ribose in 20% Dextran
up to 3 hrs of UVA lamp (visible flattening as an indicator for duration)

Representative Visual and OCT changes are shown in appended FIGS. 1-25.

From these experiments, we observed a diopter drop immediately after treatment. The diopter change was persistent at the 8-10 hours time point, showing potential for permanent vision correction.

Next, we tested the hypothesis (Hypothesis 1C) that using a femtosecond laser as the activation light source+Dextran could achieve corneal CXL, without oxygen. (Dextran is a polysaccharide.)

20% Dextran was applied for all corneas to control thickness due to focusing challenges.

Coverslip applied, oxygen independent

### Laser Treatment Specification:

Coherent Chameleon Ultra II, 755-760 nm, ˜850 mW
Pulse duration >120fs, Repetition rate 80 MHz, NA˜0.1463
Focal volume height Z: (Wz=36.6 μm)/1.376 increments, 1stlayer 25/1.376 below surface of focus sparking, 2 layers (total of 25+36.6*2˜100 μm),
Focal volume radius XY: (Wxy=1.175 μm), 3 μm grid zigzag, 5.5 mm radius half circle;
Motor speed 30 mm/s half circle treatments: ˜40 min

From these experiments we observed that using the femtosecond laser as the activation light source+Dextran significantly increased the equilibrium modulus of the cornea, but the magnitudes are small.

### Materials and Methods

Sample Preparation: A total of 9 pairs of fresh rabbit eyes were enucleated, and their epitheliums were removed. 20% Dextran solution was used to maintain the physiological thickness of the cornea. In Group1, to assess the ROS-Glycation-CXL efficiency through indentation and confocal imaging, 10 corneal buttons (Ø 3 mm) were harvested from 3 rabbits. The buttons were biopsy-punched from symmetrical positions from the OS and OD eyes as pairs such that each treated button had a non-treated control. In Group2, 9 pairs of corneal buttons harvested from 3 rabbits were CxL-ed utilizing a Dresden protocol [1] to compare traditional CxL against ROS-Glycation-CXL. The buttons were paired and characterized in the same way as Group1.

In Group3, to assess corneal flattening through topography, intact eyeballs were obtained from 3 rabbits. From each rabbit, one eye was subjected to ROS-Glycation-CXL, and the other served as a paired control. Fresh eyes were enucleated and placed into a custom-built holder. Intraocular pressure was kept at 15 mm H2O through an IV system for 3 rabbits.

Crosslinking Treatment: Corneal tissues were first exposed to 20% ribose PBS solution for 1 hour. Subsequently, ROS were generated by UV-A light irradiation at 3 mW/cm2 for an additional 1 hour for Group1 or up to 3 hours for Group3. During UV-A irradiation, a 0.15 mm thick glass coverslip was pressed on top of all corneal tissue to minimize oxygen replenishment at the anterior surface. For Group2, treated tissued were CXL-ed using a standard Dresden protocol [1]. Paired controls were not exposed to UV-A light.

Nano-Indentation: The corneal buttons were stored in PBS in a 4C fridge overnight for hydration equilibrium. Indentations of 5 μm depth using 29-35 μm diameter spherical indenters were performed on the anterior surface of the cornea buttons to acquire 25-second load-relaxation curves. For each button, the average of up to 16 indentations was used as the representative result. The equilibrium modulus and the viscoelastic ratio, defined as equilibrium modulus over instantaneous modulus, were calculated and compared [9].

Corneal Topography: For Group3 eyes, using an EyeSys Vista handheld topographer, the effective refractive power (Eff.Rp) was collected at multiple time points in a 10-hour period after eye harvest. The immediate diopter drop was defined as the difference between the initial baseline Eff.Rp and the Eff.Rp immediately after CXL. The sustained diopter drop was defined as the difference between the initial baseline Eff.Rp and the final Eff.Rp. Statistical Analysis: One-tailed paired t-tests were used for statistical analysis of Eff.Rp (Group3); two-tailed paired t-tests were used for biomechanical comparison (Group1 and 2). A p-value smaller than 0.05 was considered statistically significant.

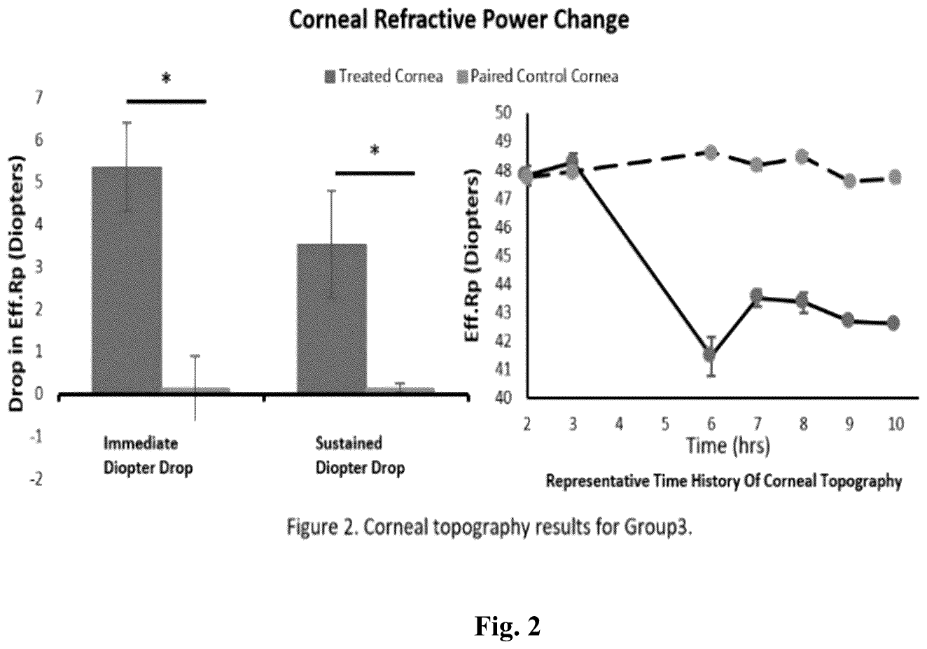

Results: In Group 1, the equilibrium modulus of ribose-CXLed corneal tissues was significantly increased (p<0.001); however, there was no change in the viscoelastic ratio (p=0.999). In Group 2, the equilibrium modulus of riboflavin-CXLed corneal tissues was significantly increased (p<0.001), and there was also a significant increase in the viscoelastic ratio (p<0.001) (FIG. 1). In Group3, there is a significant Eff.RP diopter drop for the ribose-CXLed cornea refractive power immediately after the cross-linking treatment (p=0.026), and there is also a statistically significant sustained Eff.RP diopter drop at the final time point (p=0.037) (FIG. 2).

Conclusion: The equilibrium modulus results from nano-indentation tests supported the hypothesis that the ROS accelerated, glycation-mediated CXL could achieve oxygen-independent corneal stiffening. The enhancement magnitude is comparable to the standard Dresden protocol, at least for the cornea's micro-mechanical behavior at the anterior surface. Differences in the viscoelastic ratio results between Group1 and Group2 suggested that CXL using ribose and riboflavin have different reaction mechanisms associated with collagen structure and bonding types. The immediate and sustained diopters drop after ROS-Glycation-CXL confirmed its utility in corneal flattening for non-invasive vision correction.

### Disclosure-II

Non-enzymatic cross-linking (CxL) methods, which utilize UV-A light and a photosensitizer, such as riboflavin, are used to increase corneal stiffness to treat progressive keratoconus. Such practices, like the Dresden protocol, are proven effective in clinical settings. However, they rely on the presence of oxygen and require epithelial debridement.

Advanced glycation end-products (AGE)-mediated CXL (AGE-CxL), also a non-enzymatic cross-linking mechanism via the Maillard reaction, can be a viable alternative. Under normal physiological metabolic rates, the AGE-CxL and the stiffening of tissue could take weeks or months, but the process is accelerated and potentiated by reactive oxygen species (ROS) introduced by oxidative stress conditions or by photochemical effects. The free radicals might aid the molecular rearrangement of the sugars to enable reactions between the carbohydrate functional groups and the amino acids in collagen fibrils.

We previously proposed that reshaping corneal curvature for non-invasive vision correction can be achieved if we pair mechanical deformation with simultaneous CxL. We have shown on ex vivo rabbit eyes that a localized, narrow, apical mechanical loading and simultaneous riboflavin mediated-CxL can be utilized for corneal steepening as a treatment for hyperopia [5]. However, if corneal flattening is desired for treating myopia, the apical cornea must be pressed against a wider surface, such as a coverslip; this loading profile blocks the oxygen supply, rendering the Dresden protocol ineffective. Unlike the riboflavin-mediated CxL, glycation and cross-linking of proteins by pentoses could proceed efficiently without oxygen.

Finally, ribose (150.13 Da) might penetrate corneal epithelium more easily than riboflavin (376.36 Da) and riboflavin-5-phosphate (456.3 Da) because smaller molecules have higher permeability through corneal epithelium. With its oxygen-independence mechanism, ribose-mediated CxL might be a promising candidate for the future development of efficient trans-epithelial CxL modalities.

In this study, we hypothesize that the ROS-accelerated, glycation-mediated CxL could achieve oxygen independent corneal stiffening for keratoconus treatment. In addition, we examine the simultaneous application of ROS-glycation-CxL and mechanical loading for corneal flattening toward non-invasive vision correction.

### Methods

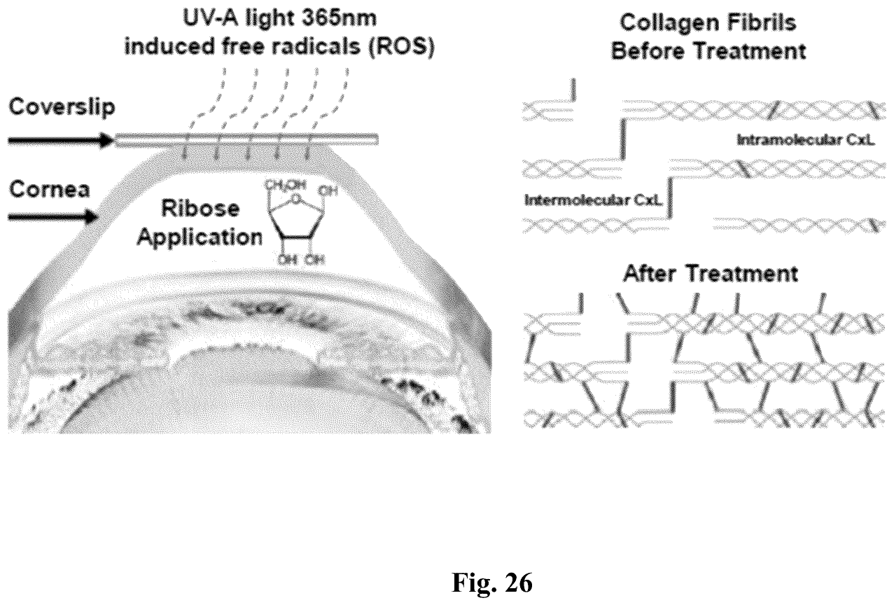

Crosslinking Treatment: Corneal tissues were first exposed to 20% Ribose PBS solution for 1 hour. Subsequently, ROS were generated by 365 nm UV-A light irradiation at 3 mW/cm\hat{ }2 for one hour for biopsy punched corneal buttons used in mechanical testing, and up to 3 hours for ex vivo rabbit eye topography. During UV-A lamp irradiation, a 0.15 mm thick glass coverslip was pressed on top of all tissues to minimize oxygen replenishment at the anterior surface (FIG. 26).

Nanoindentation: Indentations were performed on the anterior surface of the cornea buttons. The equilibrium modulus E_{∞}, instantaneous modulus Eₒ, and the viscoelastic ratio E_{∞}/Eₒ, were calculated and compared.

Corneal Topography and OCT: The EyeSys topography effective refractive power (Eff.Rp) and OCT images were collected at multiple time points in a 10-hour period after eye harvest. Diopter drop after CxL was calculated and compared relative to the baseline Eff.Rp.

According to one aspect of the disclosed subject matter, a method of treating tissue is provided. The method includes applying a biochemical agent to a surface of a target tissue and applying low intensity focused acoustic waves from an acoustic energy source toward the target tissue.

### Results

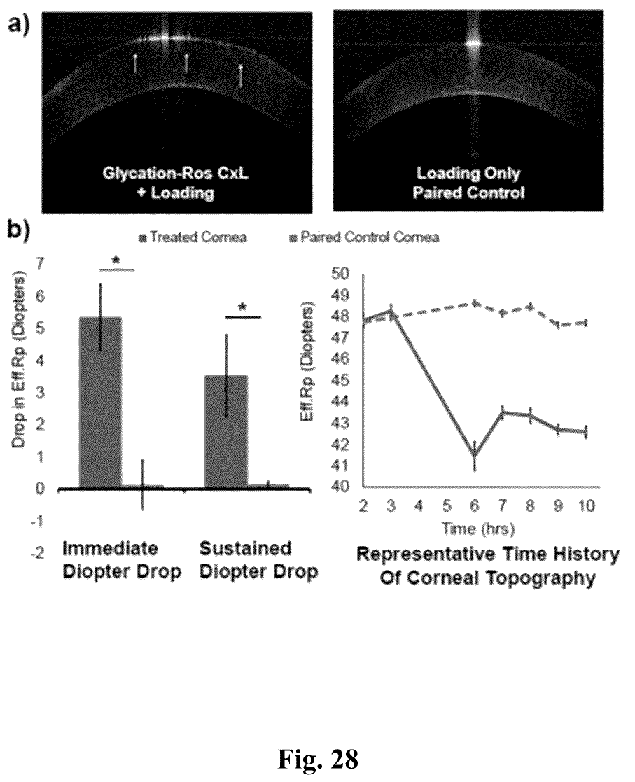

E_{∞} of ribose-CxLed corneal tissues is significantly increased; there is no significant E_{∞}/Eₒ difference. E_{∞} and E_{∞}/Eₒ of Dresden-CxL tissues are significantly increased (FIGS. 28(a)-(c)).

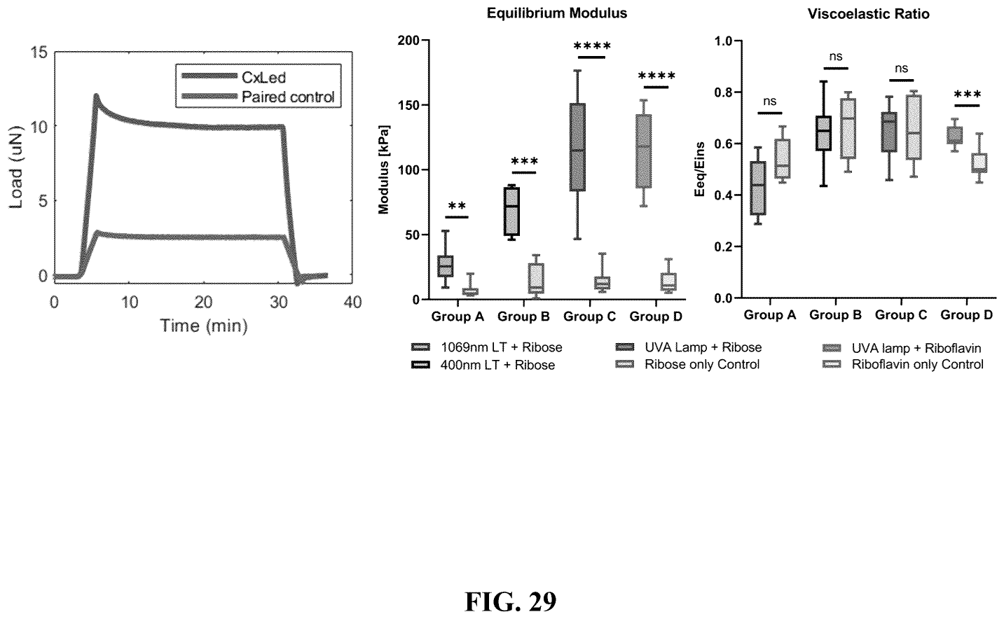

There is a significant Eff.RP diopter drop for the ribose-CxLed cornea refractive power immediately after the cross-linking treatment and the drop is stably sustained (FIGS. 29(a)-(b)).

The magnitude of achieved stiffening with ROS-Glycation-CxL is comparable to the Dresden protocol. The immediate and sustained diopter drop after ROS-Glycation-CxL combined with mechanical loading suggests its potential for non-invasive vision correction. Further investigation is warranted to assess viability of transepithelial ROS-Glycation-CxL as well as the nature of the changes in the ultrastructure of the stromal extracellular matrix.

From the foregoing, it will be appreciated that specific embodiments of the invention have been described herein for purposes of illustration, but that various modifications may be made without deviating from the scope of the invention. Accordingly, the invention is not limited except as by the appended claims.

### Disclosure-III

Although the primary goal of corneal CxL is to enhance biomechanical properties and to stop the progressive steepening of the cornea in KCN, studies have found a slight increase in visual acuity and flattening of cornea achieved. However, compared to refractive surgeries dedicated to vision correction such as LASIK, PRK, or SMILE, the viable correction range of refractive power using UVA-R5P-CxL is relatively small, and the mild diopter changes are usually observed over long investigative intervals with a possible decreased refractive precision.

Some CXL methods for spatially resolved vision correction have been investigated recently, for example using pulsed femtosecond oscillators to induce low-density plasma to create ROS from water molecules or to activate riboflavin through two-photon excitation. Notably, in these studies, the cornea was first deformed by a coverslip for accurate laser focusing and scanning, effectively crosslinking the cornea in a loaded state. Changes in diopters (3.45D ex vivo porcine/1.64D in vivo rabbits) have been reported. However, it is unclear how the additional loading and strain introduced affected the CXL and the vision corrective strength. Further, traditional UVA-R5P-CxL is not particularly compatible with extensive contact surface mechanical loading, such as apical applanating or flattening, while being crosslinked due to that technique's requirement of abundant oxygen. The application of intermittent CxL and deformation was also proven to be ineffective in promoting visual correction in monkeys with UVA-R5P-CxL after deformation through Orth-K lenses.

Another non-enzymatic collagen crosslinking pathway is advanced glycation end (AGE) product-mediated CxL via the Maillard reaction. The Maillard reaction is associated with the reaction of proteins and sugars and proceeds via the conversion of a primary amine on amino acid residues of lysine or arginine, which produce a Schiff base. The Schiff base then undergoes Amadori rearrangement, eventually producing stable AGE-mediated CxL. Evidence suggests that AGE CxLs in diabetes mellitus could protect the cornea from KC development and progression. However, glycation CxL is a slow process in physiological conditions and usually takes days of incubation for significant CxL to accumulate in vitro or ex vivo.

As described herein, ROS-Glycation-CxL can be suitable for trans-epithelial CxL in the cornea. Unique aspects of this approach include the sugar molecules' small size for ease of permeating through the epithelium and also oxygen independence. To this end, ribose, an open ring, five-carbon sugar, is a suitable agent for glycation CxL.

This disclosure describes the effect of ROS-accelerated, ribose-mediated corneal CxL; the disclosure also describes doing so while limiting the anterior cornea's access to oxygen through apical applanation with transparent coverslips. Without being bound to any particular theory or embodiment, one can hypothesize that ROS-glycation-CxL with simultaneous loading can increase the biomechanical properties of the cornea and change the corneal curvature.

### Materials and Methods

### Treatment Light Sources:

For UVA lamp irradiation (Crystal Industries, USA) at 365 nm, the distance between the UVA lamp irradiation and the plan of the sample was controlled such that an average power per area of 3 mW/cm2 was maintained, measured by a UV light meter (Sper Scientific Arizona, USA).

For 1069 nm wavelength laser treatment, a fiber oscillator (Fidelity II, Coherent, Santa Clara, CA) with a 52-femtosecond pulse duration was focused with a Zeiss Plan-NeoFluar 40× objective to produce 450 mW average power after objective with an effective numerical aperture of 0.146. A raster scan regime was performed with a 25 μm increment zig-zag pattern in a 3 mm diameter circular pattern parallel to the cornea and 10 layers of consecutive treatment planes covering the anterior 200 μm into the depth of the sample. The laser focal point was motorized by 3-dimensional translational stages and linear motors (Z825B and PT1, Thorlabs, Newton) with a speed of 2.6 mm/s and a maximum acceleration of 4 mm/s².

For 400 nm wavelength laser treatment, a wavelength tunable Ti: Sapphire oscillator (Chameleon Ultra II, Coherent, Santa Clara, CA) with a 120-femtosecond pulse duration was used at 800 nm central wavelength. The out-of-cavity 1.2 mm diameter laser beam was first expanded using a beam expander into a 3 mm diameter and focused into a BBO crystal for Type I second harmonic generation (THG800 (I)-A05-100fs, Newlight Photonics, Toronto, Canada) to generate 400 nm wavelength beam, which was filtered out of the 800 nm beam by a harmonic separator (HS10-R400/T800, Newlight Photonics, Toronto, Canada) and focused into a similar Zeiss Plan-NeoFluar 40× objective. The after-objective average power was 100 mW at an effective numerical aperture of 0.366. A similar lasing pattern point with the same zig-zag pattern and layers in depth was motorized by a hexapod (H-840.D2A-AXIS_X, Physik Instrumente, USA) at 5 mm/s and a maximum acceleration of 60 mm/s².

### Tissue and Experimental Groups:

A total of 27 pairs of fresh rabbit eyes (54 corneas) with no visible signs of corneal abnormality were obtained from a local live poultry house (La Granja Live Poultry Corporation, New York, USA) within 1 hour of sacrifice. Each eye was enucleated, and its epithelium was removed with surgical scalpels.

In Group 1 (n=12 rabbits), to assess the ROS-Glycation-CXL efficiency through indentation and confocal imaging, corneal buttons (Ø 3 mm) were biopsy punched from eyes harvested from the OS and OD eyes. Typical positions of the biopsy punch extraction are apical, nasal, temporal, inferior, and superior sites that are always symmetrical between the OS and OD eyes to avoid intra-cornea sample bias from spatial modulus difference.

For each animal, at least one button from both the OS and OD eye was chosen as the CXLed tissue to avoid inter-cornea sample bias. For each symmetrical pair of corneal buttons, one was subjected to crosslinking (either UVA-Riboflavin-CXL or ROS-Glycation-CXL), and the other served as paired control that was subject to the same condition except for light exposure. Corneal buttons were exposed to 20% ribose (Sigma-Aldrich Inc., USA) PBS (HyClone/Cytiva, UCA) for 1 hour in groups 1A, 1B, and 1C (n=3 each). 1A and 1B were then irradiated with femtosecond laser with 450 mW at 1069 nm and 250 mW at 400 nm, respectively. 1C was exposed to a 365 nm UVA lamp for 1 hr. The corneal anterior surfaces were pressed with a glass coverslip to minimize oxygen replenishment. 1D (n=3) was CXLed following the Dresden protocol as positive controls using the 0.1% riboflavin-5-phosphate (R5P) in 20% dextran 500 (Sigma-Aldrich Inc., USA) solution. All corneal buttons were left in the 4C fridge in PBS to reach hydration equilibrium overnight and mechanically tested the next day.

In Group2 (n=15 rabbits), to assess corneal flattening, 24 intact eyeballs (12 rabbits) were enucleated and placed into a custom-build holder such that one eye was subject to ROS-Glycation-CXL, and the other served as a paired control. After 1 hr incubation in 20% ribose PBS, all apical eyeballs were pressed with a coverslip until the contact between the cornea and the coverslip reached approximately 3 mm in diameter until the treatment finished. Group 2A's (n=6 pairs) treated eyes were exposed to 1.5 hours of UVA lamp irradiation, whereas Group 2 B's (n=6 pairs) treated eyes were exposed to 3 hours of UVA lamp irradiation. Intraocular pressure was kept at 18-20 mm H₂O through an IV system with needles connecting at the posterior side of the eyeball. In order to investigate the mechanical difference between the 1.5 hrs and 3 hrs irradiation groups, Group 2C's (n=3 pairs) corneas went through similar preparations as Group 1 corneas did such that the two treatments with different irradiation times were applied to the pair of eyes from the same animal.

### Topography and OCT:

Effective refractive power (Eff. RP) was measured using a handheld topographer (EyeSys Vista, Teas, USA) for Group 2A and 2B eyes. The first point measured at 0.5-1 hour represents the baseline initial refractive power after stabilization at the controlled intraocular pressure before any alterations. The second point measured at 2 hours represents the refractive power after the ribose solution application. The third point at 3.5 hours for Group 2A or 5 hours for Group 2B represents refractive power after ROS-Glycation-CXL or loading for control eyes. The fourth, fifth, and sixth points represent the refractive power development, with the seventh point being the final refractive power of each cornea at approximately 10 hours after sacrifice. Multiple topographies were taken at each point, and the average was calculated as the representative refractive power.

The corneas' cross-section and apical thickness were measured using an optical coherence tomographer (OQ LabScope Lumedica, North Carolina, USA) at the same time points described above. The thickness of the corneas was first segmented in ImageJ and calculated using a constant pixel-to-length conversion factor.

### Multiphoton Imaging:

The autofluorescence and second harmonic generation (SHG) signals of the corneal tissues in Group A were investigated to reveal the presence of CxLs and structural changes in collagenous ECM after hydration equilibrium in PBS on the same night of individual treatment days. Imaging was performed with a Nikon TI Eclipse inverted microscope using a Ti: Sapphire laser Chameleon Ultra II at 860 nm as the light source and a Nikon Apo LWD 20× objective for focusing. The emission filter was set at the FITC channel (475-650 nm, peak 525 nm) for autofluorescence signal collection and at the DAPI channel (400-600 nm, peak at 457 nm) for SHG signal collection. The XY scanning area for each sample was 635 μm by 635 μm, and Z scanning was 100 μm deep with 10 μm increments (stack of 11 images per sample). The summation of the average grey value processed in Image J at each layer was used as the representative signal strength for each sample.

### Nanoindentation:

After overnight incubation in PBS at 4C to reach equilibrium hydration, corneal buttons in Group A were allowed to return to room temperature the next morning after the respective treatment days while glued to the bottom of a petri-dish with the anterior surface of the corneal button facing upward. A displacement-controlled indenter (Piuma, Optics 11, Amsterdam, The Netherlands) was used to perform micro-indentations on top of the sample surface submerged in PBS; for each sample, multiple tests were performed following an array of 16 equally spaced (4×4) contact points with a spacing of 100 μm to 150 μm, covering roughly a 0.4×0.4 mm² area at the center of each corneal button. A spherical glass probe with a 34 μm radius and 0.2 N/m stiffness was used for samples in Group 1A; another spherical glass probe with a 25.5 μm radius and 4.09 N/m stiffness was used for Group 1B, 1C, 1D, and 2C. The indentation process at each contact point was comprised of three consecutive steps of loading Piezo movement of 5 μm for 2 seconds, holding time of 5 s, and unloading for 2 seconds. The load-relaxation curves obtained after indenting each point were then processed using a viscoelastic model to obtain equilibrium modulus and viscoelastic ratio following a fitting algorithm developed by Oyen.

### Results

### Micro-Indentation

The equilibrium modulus significantly increased after ROS-Glycation-CxL for all three light sources. While the ribose-only control corneal tissues universally had an average modulus around 10 kPa, the equilibrium modulus of crosslinked corneas significantly increased to 25 kPa for 1069 nm laser treatment in Group 1A (p<0.01), 60 kPa for 400 nm laser treatment in Group 1B (p<0.001), and 100 kPa for 365 nm UVA lamp treatment for Group 1C (p<0.0001). The positive control group with cornea crosslinked with the Dresden protocol also significantly increased equilibrium modulus to 100 kPa (p<0.0001).

No significant changes in the time-dependent viscoelastic ratio were found for all three Group 1 A, B, and C between the ROS-Glycation-CxLed corneas and paired controls. However, corneal tissues CxLed with the Dresden protocol have a more elastic response compared to the R5P-exposed-only paired controls (p<0.001) (FIG. 29).

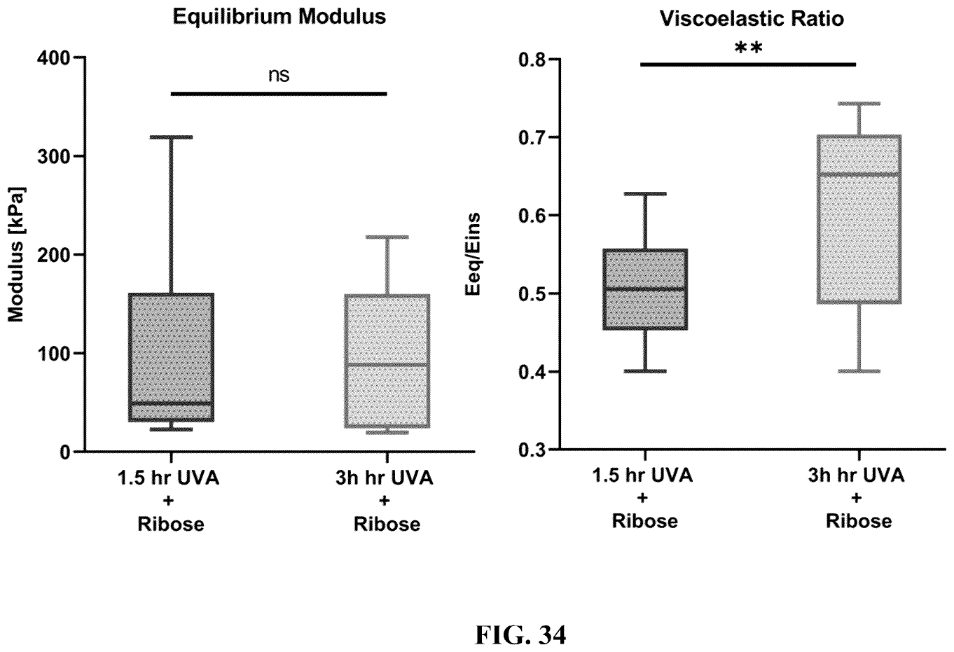

For Group 2C, between the 1.5 hours and 3 hours 365 nm UVA lamp irradiation for ROS-Glycation-CxL treatment, no significant difference was found in the equilibrium modulus. Interestingly, 3-hour 365 nm UVA irradiation yielded a significantly higher viscoelastic ratio (p<0.01) (FIG. 34).

### Multiphoton Imaging

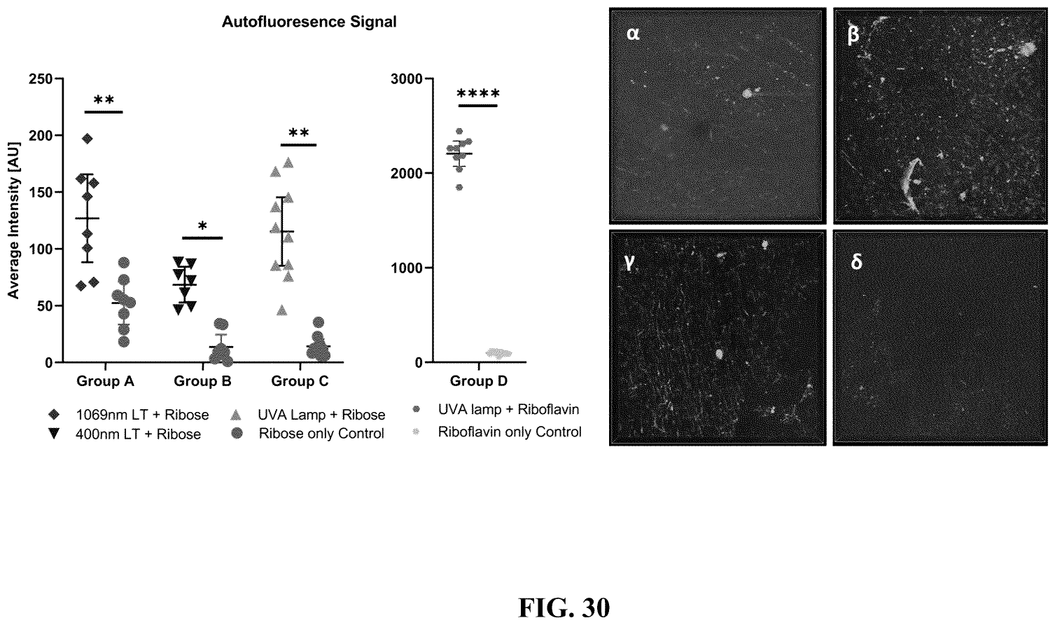

The autofluorescence signal intensity of the anterior 100 μm corneal tissues increased significantly for all three ROS-Glycation-CxL groups with 1060 nm laser treatment in Group1A (p<0.01), 400 nm laser treatment in Group 1B (p<0.05), and 365 nm UVA lamp treatment in Group 1C (p<0.01) compared to the paired ribose only control, albeit the small difference in magnitude compared to the increase in Dresden protocol CxLed tissues from R5P only control (p<0.0001). An apparent signal increase can be observed in laser scanning tracks for 400 nm laser treatment but not for 1060 nm laser treatment (FIG. 30).

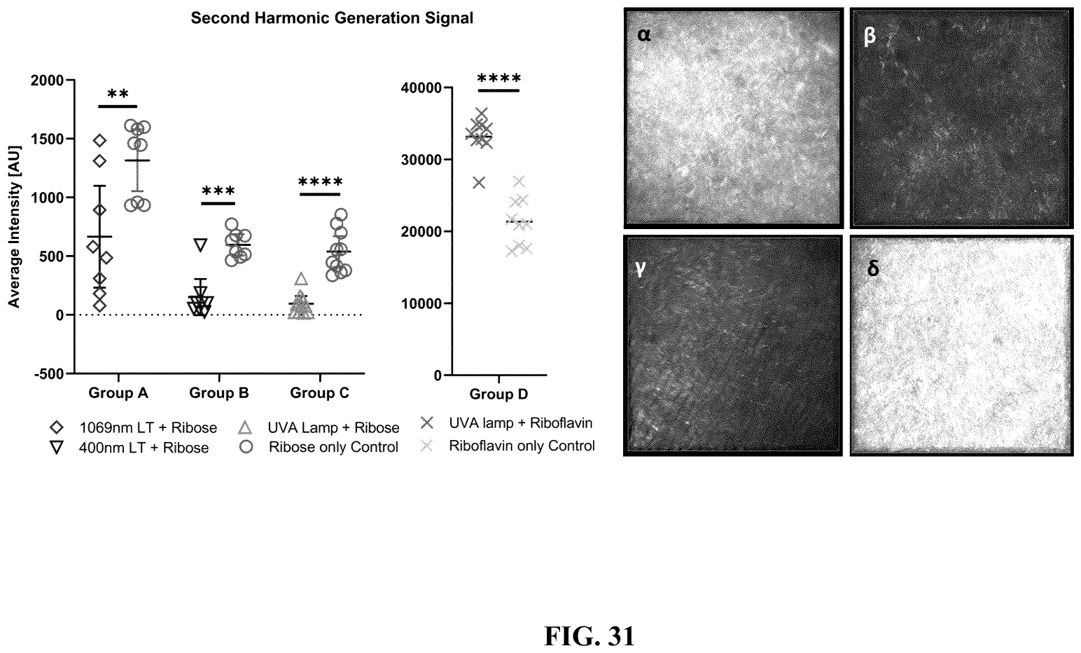

The second harmonic generation signal intensity of the anterior 100 μm corneal tissues decreased significantly for all three ROS-Glycation-CxL groups with 1060 nm laser treatment in Group1A (p<0.01), 400 nm laser treatment in Group 1B (p<0.001), and 365 nm UVA lamp treatment in Group 1C (p<0.0001) compared to the ribose only control. Conversely, Dresden protocol CxLed corneas showed increased SHG signal compared to R5P exposed paired control. A clear decrease of signals can be observed in laser scanning tracks for 400 nm laser treatment but not for 1060 nm laser treatment (FIG. 31).

### Corneal Anterior Surface Topography

The averaged time histories of corneal topography showed a sustained drop of refractive power after recovering slightly from coverslip flattening in all ROS-Glycation-CxLed eyes in Groups 2A and B. The paired control eyes under 3-hour loading experienced a more obvious drop in diopters compared to 1.5-hour loading, which had a close to zero response to coverslip flattening. However, all controls recovered to the baseline refractive power at the 10-hour time point.

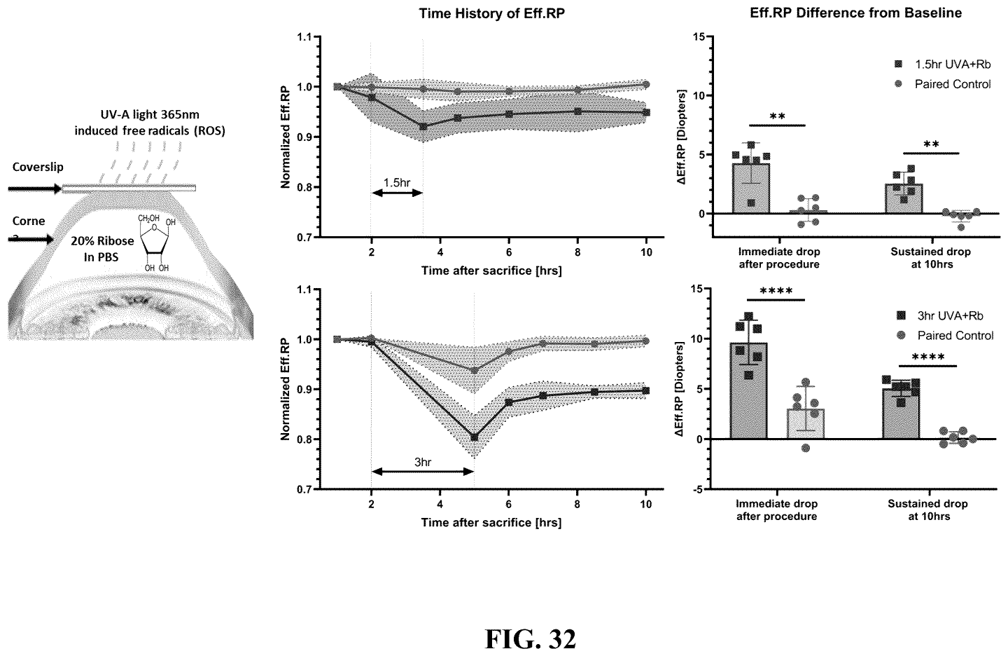

The average effective refractive power difference was calculated for each eye by subtracting the baseline diopter measured at the 0.5-hour timepoint from the third timepoint as the “immediate diopter drop” and the final timepoint as the “sustained diopter drop.” There is a significant decrease in refractive power immediately after 1.5 hr 365 nm UVA lamp irradiation of 5 diopters (p<0.01) in Group 2A and immediately after 3 hr irradiation of 10 diopters (p<0.0001) in Group 2B while simultaneously flattened under the coverslip compared to the control eyes without light exposure. At the 10 hr time point, the decrease in refractive power slightly recovered but stabilized at 2.5 diopters (p<0.01) in Group 2A and 5 diopters (p<0.0001) in Group 2B (FIG. 32). In general, the effective refractive power changes introduced by the 3 hr treatment were more pronounced compared to the 1.5 hr treatment changes.

### Optical Coherence Tomography and Corneal Apical Thickness

Representative OCT pictures at the apical cornea at the initial, third, and final time points recorded increased cornea thickness for all eyes under the IV system's controlled, artificial intraocular pressure. Notably, the central flattening of the cornea by the coverslip produced slight curvature changes at the central apex approximately in the size of the cornea-coverslip contact area around 3 mm diameter for samples exposed to 365 nm UVA light.

The difference in the average apical thickness was calculated for each eye by subtracting the pixel length measured at the 0.5-hour time point from the third time point as the “immediate thickness change” and from the final time point as the “sustained thickness change”. For Group 2B, the simultaneous coverslip flattening and 3 hr ROS-Glycation CxL treatment produced a significant 400 nm thickness increase immediately after treatment compared to the loading-only control (p<0.05). There was no significant difference in sustained thickness change between the treated and control eyes as all eyes had approximately above 200 μm thickness increase compared to the baseline. For Group 2A, the simultaneous coverslip flattening and 1.5 hr ROS-Glycation CxL treatment had no significant thickness change difference immediately after treatment compared to the loading-only control. Similarly, there was no significant difference in sustained thickness change between the treatment and control eyes as all eyes had approximately above 100 μm thickness increase compared to the baseline. The thickness changes introduced by the 3 hr treatment were generally more pronounced than the 1.5 hr treatment changes.

### DISCUSSION

Presented here is an investigation of the synergistic effects of ROS and non-enzymatic glycation using ribose on crosslinking rabbit corneal stroma tissues. As a load-bearing tissue, the cornea accounts for 80% of the visual power in the ophthalmic system, and the biomechanical strength of the stroma is crucial for maintaining appropriate curvature for light refraction into the retina under intraocular pressure. Prevalent diseases such as KCN have been shown to cause a decrease in the mechanical strength in corneal stroma where UVA-Riboflavin crosslinking is adapted as a treatment to retard disease progression in astigmatism and keratoconus. In this study, non-enzymatic glycation was used as an alternative crosslinking method to change the mechanical and refractive properties of the cornea.

Though glycation itself is able to generate crosslinking, in physiological temperature, this process usually takes days of incubation, and sugars like ribose and glucose alone are relatively ineffective crosslinkers. In order to expedite the process of AGE-mediated crosslinking, it has been shown that oxidative stress and the introduction of photo-generated ROS are catalytic to the CxL reaction.

Here, we used three different methods to create ROS inside the corneal stroma. First, previous study has shown that a tightly focused oscillator laser beam at the infrared range (1059.6 nm) with pulse energy in the nano-joule range and pulse duration in the femtosecond range can generate low-density plasma-induced ROS from interstitial water at the laser focal volume; here, we used a slightly different fiber oscillator at 10 with the intention to produce ROS in a similar mechanism. Second, a UV-A lamp with a central wavelength of 365 nm was chosen as it is a typical light source used in the traditional corneal crosslinking protocol using riboflavin-5-phosphate, and free-radical species are generated by exposure of collagen to UV light. Finally, we converted an 800 nm Ti: Sapphire oscillator beam with femtosecond pulse duration to 400 nm through the Type I SHG mechanism to investigate the effect of ROS generated by UVA/blue light range ultrafast laser.

As pentoses were shown to be oxygen independent in the glycation process, we minimized oxygen accessibility in the CxL process. Due to the coverslip pressing against the anterior surface of the cornea, and the surrounding ribose PBS solution, oxygen availability during UVA irradiation was limited. To further limit oxygen exposure, Ribose solution was intentionally not replenished every five minutes (like in the Dresden protocol) during UVA exposure; however, to make sure that there were enough sugar molecules, we compensated with a high concentration of ribose (20%) in the PBS solution.

Measurements of the equilibrium modulus and the time-dependent viscoelastic ratio at multiple locations within a prescribed indentation matrix after ROS-Glycation-CxL were performed. Micro indentation tests with a 5 μm indentation were chosen as the mechanical test to focus on the mechanical behavior of the treatment region: for laser treatments, the treatment volume focused at only the anterior 200 μm; for UVA lamp treatments, previous studies have also shown that the Dresden protocol had a demarcation line around the 200 μm from the anterior surface. For all three ROS generation methods to accelerate ribose-based glycation, the average equilibrium modulus of crosslinked tissue is significantly higher than the ribose-exposed-only, coverslip-pressed control. Out of the three groups, the 1060 nm laser-treated group stiffened the smallest amount at 450 mW at 26.7 kPa, ˜3.8 fold of control's modulus, followed by the 400 nm laser-treated group at 100 mW at 63.2 kPa, ˜4.6 fold of controls modulus. Notably, one-hour irradiation of 365 nm UVA lamp treatment yielded a modulus of 115.3 kPa, ˜8.2 fold of paired control modulus. This magnitude of equilibrium modulus change is on par with the UVA-R5P treatment, establishing the strong stiffening potential of the ROS-Glycation-CxL.

None of the ROS-Glycation-CxL treatments produced a significant difference in the time-dependent viscoelastic ratio difference from the control samples. In contrast, the UVA-R5P-CxL produced a more elastic response in the tissue, meaning there is a smaller difference between the initial contact force and equilibrium force in the load-relaxation curve. Without being bound to any particular theory, this difference between the two crosslinking mechanisms may be due to different changes and crosslinking sites in the stroma ultrastructure, which determines how the collagen lamella and fibrillar structure crimp, rotate and stretch under loading for the micro indentation test.

Collagen autofluorescence has been used as another indicator for the formation of crosslinking. It has been shown that for UVA-R5P-CxL, the signal intensity of collagen autofluorescence positively correlates with crosslinking efficiency and biomechanical enhancement. Additionally, after infrared femtosecond laser ROS-induced crosslinking, collagen autofluorescence signal has also been reported to increase in the raster-canned region. For all three ROS generation methods, after ROS-Glycation-CxL, the autofluorescence signal intensity, as the sum of grey values in the imaged volume at the anterior 100 μm stroma, slightly increased compared to the paired control (FIG. 31). For the positive control group, the autofluorescence signal of the Dresden protocol crosslinked group has also increased as expected. However, the magnitude of autofluorescence change is different. Without being bound to any particular theory, one may hypothesize that the emission window of used in this study, between 475 nm to 650 nm, covers the flavoproteins (emission peak at 550 nm) that originate from the riboflavin-5-phosphate induced crosslinking but does not cover the pentosidine-type crosslinking (emission peak at 380 nm) at that might from in glycation process.

The second harmonic generation signal captured in corneal stroma comes from the interaction of light with non-centrosymmetric structures in collagen type I. Due to this underlying intrinsic origin, the signal is highly sensitive to collagen fibril and fiber ultrastructure, which is subject to change after CxL. We observed a statistically significant increase in the summary of grey value at the most anterior 100 μm of UVA-R5P-CxLed cornea tissues. In contrast, we observed statistically significant decreases of SHG signal in treated tissues after ROS-Glycation-CxLed for all three light sources. Interestingly, for the 365 nm UVA-lamp irradiated and 400 nm laser-treated groups with higher stiffening, the SHG signal decreased more than the 1069 nm laser-treated group with less mechanical strength modification. We suspect the different trends observed in SHG signal alterations after CxL are partially due to how ROS-Glycation changed the original collagenous ECM structure in a different mechanism than the UVA-Riboflavin. Again without being bound to any particular theory or embodiment, it is also possible that compared to Riboflavin-5-Phosphate, ribose might not have had equally effective protection against ultraviolet light, and there was potentially some fibrillar scission or unraveling due to highly concentrated exposure to ROS.

Overall, we observed laser scanning patterns in the 400 nm group but not in the 1060 nm group. Without being bound to any particular theory, there may be two reasons behind this. First, though the average power at 1060 nm (450 mW) is higher than the power at 400 nm (100 mW), due to out-of-cavity beam expansion required for the SHG crystal, the 400 nm beam path had a much higher effective numerical aperture, resulting in a much more focused beam. Second, it has been shown that high UV, blue wavelength is much more effective at collagen manipulation in the corneal tissues compared to the IR or near-IR wavelength. This trend of increased autofluorescence and decreased SHG signal coincidently agrees with other studies using femtosecond oscillator's manipulation with collagenous tissue in similar studies.

In order to investigate ROS-Glycation-CxL's potential for introducing vision correction and changing anterior corneal curvature, the 365 nm UVA lamp irradiation treatment group with the highest equilibrium modulus enhancing capability was chosen. We have previously investigated the effect of simultaneous loading and UVA-R5P-CxL on corneal curvature change for a steepening effect, where the central 1.5 mm diameter cornea was blocked from light exposure, and the cornea was in a tensile loading state during CxL. However, to create a flattening effect, the central cornea must be pressed against a transparent surface such as a coverslip so that the mechanically loaded surface is still crosslinked. This configuration is challenging for UVA-R5P-CxL because the process is oxygen-dependent, but continuous mechanical flattening blocks oxygen access. Alternatively, ribose-induced oxygen-independent ROS-Glycation-CxL is ideal for verifying the simultaneous flattening and crosslink effect on corneal curvature. Two irradiation time was chosen to test the flexibility of the vision correction magnitude. Data from corneal topography shows a sustained five diopter drop from 3-hour treatments and a sustained 2.5 diopter drop from 1.5-hour treatments. Mechanical flattening by the coverslip also had an observable effect on the decrease of effective refractive power, but this effect can be seen to have fully recovered by the final time point. From OCT, all corneas experienced a significant increase in thickness, possibly due to swelling by the IV system, constant replenishment of PBS on the anterior surface for the artificial tear film creation required for topography, and mechanical flattening.

Indentation results comparing 1.5-hour and 3-hour treatment groups also showed that there was no significant difference in mechanical properties on the micro-scale at the anterior corneal surface; however, the change in time-dependent viscoelastic properties might be the reason for the magnitude difference on the stabilized time history evolution of corneal curvatures.

It should be understood that the experiments described herein are illustrative only and do not limit the scope of the present disclosure or the appended claims. For example, one can use a sugar concentration that differs from those used in the exemplary experiments. One can also use repeated, shorter durations of UVA lamp irradiation. Further, because laser oscillator treatments effectively enhanced the corneal mechanical properties, 400 nm blue light laser treatment can be useful as a ROS distribution model since it only required a relatively low power to be efficient in CxL and may demonstrate a specially resolved treatment pattern for more complexed C×L patterns. One can use a dextran solution to control the swelling and hydration content of the cornea to improve corneal edema caused by flattening and glycation.

In comparison to existing methods, the disclosed technology (1) is not wavelength dependent during photo-activation; (2) has better epithelium permeability if the applied glycation agent is small (e.g., ribose) thereby reducing or even eliminating the need for epithelium removal; and (3) can be oxygen independent due to diverse mechanisms of the Millard reaction. Thus, the disclosed ROS-glycation-CxL technology provides utility in transepithelial corneal crosslinking procedures.

### Aspects

The following Aspects are illustrative only and do not limit the scope of the present disclosure or the appended claims. Any part or parts of any one or more Aspects can be combined with any part or parts of any one or more other Aspects.
Aspect 1. A method of treating a tissue of a cornea, the method comprising: contacting the tissue with a sugar; and illuminating the tissue with any one or more of UV-A light or a femtosecond laser, the illuminating being performed under conditions sufficient to give rise to crosslinking within the tissue.
Aspect 2. The method of Aspect 1, wherein the sugar comprises a complex sugar. Dextran is an example complex sugar, although other complex sugars are also suitable.
Aspect 3. The method of Aspect 1, wherein the sugar comprises a simple sugar. Simple sugars include, for example, 5-carbon sugars (arabinose, ribose, xylose), 6-carbon sugars (glucose, fructose, mannose, galactose), glyceraldehyde, and methylglyoxal.
Aspect 4. The method of any one of Aspects 1-3, wherein the sugar is present in a solution.
Aspect 5. The method of Aspect 4, wherein the sugar is present in the solution at from about 5 to about 50 wt %.
Aspect 6. The method of Aspect 5, wherein the sugar is present in the solution at from about 10 to about 25 wt %.
Aspect 7. The method of any one of Aspects 1-6, wherein the UV-A light has a wavelength of 365 nm.
Aspect 8. The method of any one of Aspects 1-7, wherein the cornea is characterized as free of epithelial debridement.
Aspect 9. The method of any one of Aspects 1-8, wherein the tissue contacts the sugar for up to about 2 hours.
Aspect 10. The method of any one of Aspects 1-9, wherein the illuminating is performed for up to about 3 hours.
Aspect 11. The method of Aspect 10, wherein the illuminating is performed for about 1 hour.
Aspect 12. The method of Aspect 10, wherein the illuminating is performed for about 3 hours.
Aspect 13. The method of any one of Aspects 1-12, comprising illuminating the tissue with UV-A light.
Aspect 14. The method of Aspect 13, wherein the UV-A light is applied at about 3 mW/cm2.
Aspect 15. The method of any one of Aspects 1-12, comprising illuminating the tissue with a femtosecond laser.
Aspect 16. The method of any one of Aspects 1-15, further comprising restricting oxygen replenishment of the tissue.
Aspect 17. The method of Aspect 16, wherein the restricting comprises superposing a barrier over the cornea.
Aspect 18. The method of Aspect 17, wherein the barrier contacts the cornea.
Aspect 19. The method of any one of Aspects 17-18, wherein the barrier is essentially transparent to at least one of UV-A light and a femtosecond laser.
Aspect 20. The method of any one of Aspects 1-19, further comprising application of a mechanical loading to the cornea.
Aspect 21. The method of Aspect 20, wherein the mechanical loading is applied during the illuminating.
Aspect 22. The method of any one of Aspects 20-21, wherein the mechanical loading is effected by an element that contacts the tissue, and further wherein at least some of the crosslinking is effected in a region of the tissue illuminated by illumination that passes through the element.
Aspect 23. The method of any one of Aspects 20-22, wherein the mechanical loading at least partially applanates the cornea.
Aspect 24. The method of any one of Aspects 20-22, wherein the mechanical loading steepens the cornea.
Aspect 25. The method of any one of Aspects 1-24, wherein the cornea is of a subject having at least one of a refractive error or keratoconus.
Aspect 26. The method of Aspect 25, wherein the cornea is of a subject having keratoconus.
Aspect 27. The method of Aspect 26, further comprising application of a mechanical loading to the cornea.
Aspect 28. The method of Aspect 27, wherein the method is performed so as to effect a change in the shape of the cornea.
Aspect 29. The method of Aspect 25, wherein the cornea is of a subject having at least one refractive error.
Aspect 30. The method of Aspect 29, wherein the at least one refractive error is any one or more of astigmatism, myopia, hyperopia, or stigmatism.
Aspect 31. A method of treating a tissue of a cornea, the method comprising: restricting oxygen replenishment of the tissue; contacting the tissue with a sugar; and illuminating the tissue with at least one of UV-A light or a femtosecond laser, the illuminating being performed under conditions sufficient to give rise to crosslinking within the tissue.
Aspect 32. The method of Aspect 31, wherein the cornea is characterized as free of epithelial debridement.
Aspect 33. The method of any one of Aspects 31-32, further comprising application of a mechanical loading to the cornea.
Aspect 34. The method of Aspect 33, wherein the mechanical loading at least partially applanates the cornea.
Aspect 35. The method of Aspect 34, wherein the mechanical loading steepens the cornea.
Aspect 36. The method of any one of Aspects 31-35, wherein the restricting comprises superposing a barrier over the cornea.
Aspect 37. The method of Aspect 36, wherein the barrier contacts the cornea.
Aspect 38. The method of any one of Aspects 31-37, wherein the cornea is of a subject having at least one of a refractive error or keratoconus.
Aspect 39. The method of Aspect 38, wherein the cornea is of a subject having keratoconus.
Aspect 40. The method of Aspect 39, further comprising application of a mechanical loading to the cornea.
Aspect 41. The method of Aspect 40, wherein the method is performed so as to effect a change in the shape of the cornea.
Aspect 42. The method of Aspect 38, wherein the cornea is of a subject having at least one refractive error.
Aspect 43. The method of Aspect 42, wherein the at least one refractive error is any one or more of astigmatism, myopia, hyperopia, or stigmatism.
Aspect 44. A method of treating a collagenous tissue, the method comprising: contacting the tissue with a sugar; and illuminating the tissue with any one or more of UV-A light or a femtosecond laser, the illuminating being performed under conditions sufficient to give rise to crosslinking within the tissue.
Aspect 45. The method of Aspect 44, wherein the collagenous tissue is any one or more of a cornea, skin, or cartilage.

Other suitable collagenous materials include, for example, collagenous reproductive tissues (for example, uterus and cervix), musculoskeletal tissues (for example, ligament and tendon), ophthalmological tissues (for example, sclera and lens), dentin bonding, cardiovascular tissue. The disclosed technology can be applied to, for example, strengthening of wounds after surgeries or any applications of stitches, as well as bonding tissue constructs to actual tissues for repair. As but one example, one can add a filler into a torn ligament and then crosslink the two to form a bond between the two.
Aspect 46. The method of Aspect 45, wherein the collagenous tissue is a cornea.

4897-8653-0099.1-31-·
Aspect 47. The method of Aspect 45, wherein the collagenous tissue is skin.
Aspect 48. The method of Aspect 45, wherein the collagenous tissue is cartilage.
Aspect 49. The method of any one of Aspects 45-48, further comprising application of a mechanical loading to the cornea.
Aspect 50. The method of Aspect 49, wherein the mechanical loading is applied during the illuminating.
Aspect 51. The method of any one of Aspects 49-50, wherein the mechanical loading is effected by an element that contacts the tissue, and further wherein at least some of the crosslinking is effected in a region of the tissue illuminated by illumination that passes through the element.
Aspect 52. The method of any one of Aspects 49-50, wherein the mechanical loading changes a shape of the tissue.
Aspect 53. The method of Aspect 52, wherein the mechanical loading effects any one or more of a flattening, an indenting, or a steepening of the tissue.
Aspect 54. The method of Aspect 53, wherein the mechanical loading effects a flattening of the tissue.
Aspect 55. The method of Aspect 53, wherein the mechanical loading effects a steepening of the tissue.
Aspect 56. The method of any one of Aspects 49-50, wherein the mechanical loading steepens the tissue.
Aspect 57. The method of any one of Aspects 44-56, further comprising restricting oxygen replenishment of the tissue.
Aspect 58. The method of Aspect 57, wherein the restricting comprises superposing a barrier over the tissue.
Aspect 59. The method of Aspect 58, wherein the barrier contacts the tissue.
Aspect 60. The method of any one of Aspects 58-59, wherein the barrier is essentially transparent to at least one of UV-A light and a femtosecond laser.

It should be understood that although the disclosed technology is illustrated by refence to the cornea, the disclosed technology is not limited to corneal application. The disclosed technology can be applied to effect crosslinking in essentially any collagenous tissue, including skin and cartilage.

1. A method of treating a tissue of a cornea, the method comprising:
contacting the tissue with a sugar; and
illuminating the tissue with any one or more of UV-A light or a femtosecond laser, the illuminating being performed under conditions sufficient to give rise to crosslinking within the tissue.

2. (canceled)

3. (canceled)

4. The method of claim 1, wherein the sugar is present in a solution, the sugar optionally present in the solution at from about 5 to about 50 wt %.

5. (canceled)

6. (canceled)

7. The method of any one of claim 1, wherein any one or more of (1) the tissue is illuminated with UV-A light has-having a wavelength of 365 nm, (2) the cornea is characterized as free of epithelial debridement, (3) the tissue contacts the sugar for up to about 2 hours, and (4) the illuminating is performed for up to about 3 hours.

8. (canceled)

9. (canceled)

10. (canceled)

11. (canceled)

12. (canceled)

13. The method of claim 1, comprising illuminating the tissue with UV-A light, the UV-A light optionally applied at about 3 mW/cm2.

14. (canceled)

15. The method of claim 1, comprising illuminating the tissue with a femtosecond laser.

16. The method of claim 1, further comprising restricting oxygen replenishment of the tissue, the restricting optionally comprising superposing a barrier over the cornea, the barrier optionally contacting the cornea.

17. (canceled)

18. (canceled)

19. The method of claim 1716, the restricting comprising superposing a barrier over the cornea, the barrier being essentially transparent to at least one of UV-A light and a femtosecond laser.

20. The method of claim 1, further comprising application of a mechanical loading to the cornea, the mechanical loading optionally being applied during the illuminating.

21. (canceled)

22. The method of claim 20, wherein the mechanical loading is effected by an element that contacts the tissue, and further wherein at least some of the crosslinking is effected in a region of the tissue illuminated by illumination that passes through the element.

23. (canceled)

24. (canceled)

25. The method of claim 1, wherein the cornea is of a subject having at least one of a refractive error or keratoconus.

26. (canceled)

27. (canceled)

28. The method of claim 20, wherein the method is performed so as to effect a change in the shape of the cornea.

29. The method of claim 25, wherein the cornea is of a subject having at least one refractive error, the refractive error optionally being any one or more of astigmatism, myopia, hyperopia, and stigmatism.

30. (canceled)

31. A method of treating a tissue of a cornea, the method comprising:
restricting oxygen replenishment of the tissue;
contacting the tissue with a sugar; and
illuminating the tissue with at least one of UV-A light or a femtosecond laser, the illuminating being performed under conditions sufficient to give rise to crosslinking within the tissue,
optionally, the cornea being of a subject having at least one of a refractive error or keratoconus, and
optionally, the cornea is characterized as free of epithelial debridement.

32. (canceled)

33. The method of claim 31, further comprising application of a mechanical loading to the cornea, the mechanical loading optionally at least partially applanating the cornea or steepening the cornea.

34. (canceled)

35. (canceled)

36. The method of claim 31, wherein the restricting comprises superposing a barrier over the cornea, the barrier optionally contacting the cornea.

37. (canceled)

38. (canceled)

39. (canceled)

40. The method of claim 31, further comprising application of a mechanical loading to the cornea, the method optionally performed so as to effect a change in the shape of the cornea.

41. (canceled)

42. (canceled)

43. (canceled)

44. A method of treating a collagenous tissue, the method comprising:
contacting the tissue with a sugar; and
illuminating the tissue with any one or more of UV-A light or a femtosecond laser, the illuminating being performed under conditions sufficient to give rise to crosslinking within the tissue.

45. The method of claim 44, wherein the collagenous tissue is any one or more of skin, cartilage, reproductive tissue, musculoskeletal tissue, ophthalmological tissue, dentin, or cardiovascular tissue.

46. (canceled)

47. The method of claim 45, wherein the collagenous tissue is skin.

48. The method of claim 45, wherein the collagenous tissue is cartilage.

49. (canceled)

50. The method of claim 49, wherein the mechanical loading is applied during the illuminating, the mechanical loading optionally effected by an element that contacts the tissue, and further wherein at least some of the crosslinking is effected in a region of the tissue illuminated by illumination that passes through the element.

51. (canceled)

52. The method of claim 50, wherein the mechanical loading changes a shape of the tissue.

53. The method of claim 52, wherein the mechanical loading effects any one or more of a flattening, an indenting, or a steepening of the tissue.

54. (canceled)

55. (canceled)

56. (canceled)

57. The method of claim 44, further comprising restricting oxygen replenishment of the tissue.

58. The method of claim 57, wherein the restricting comprises superposing a barrier over the tissue, the barrier optionally contacting the tissue.

59. (canceled)

60. The method of claim 58, wherein the barrier is essentially transparent to at least one of UV-A light and a femtosecond laser.
